SEQUENCE LISTING (4 sequences)

SEQ ID NO: 1 - DNA, 32 bp, artificial
  primer
gagagagcggccgccacccagaaacgctggtg

SEQ ID NO: 2 - DNA, 39 bp, artificial
  primer
agacagagctcgcatgctgagtaaacttggtctgacagt

SEQ ID NO: 3 - DNA, 31 bp, artificial
  primer
ggatccgcatgcgcactctcggcagaccatc

SEQ ID NO: 4 - DNA, 27 bp, artificial
  primer
ggcggccgctaggacttggcatttgtg

(19) United States
(12) Patent Application Publication
(10) Pub. No.: US 2011/0319398 A1
(43) Pub. Date: Dec. 29, 2011

(54) INHIBITION OF YOPS TRANSLOCATION

(75) Inventor: Joan Mecsas, Needham, MA (US)

(73) Assignee: TUFTS UNIVERSITY, Boston, MA (US)

(21) Appl. No.: 13/150,495

(22) Filed: Jun. 1, 2011

Related U.S. Application Data
(60) Provisional application No. 61/350,990, filed on Jun. 3, 2010.
(60) Provisional application No. 61/377,458, filed on Aug. 26, 2010.

(51) Int. Cl. A61K 31/5415; C07D 279/08; C07D 491/044; C07D 231/38; C07D 513/04; C07D 417/14; C07D 403/10; C07D 413/12; A61K 31/4965; A61K 31/4985; A61K 31/415; A61K 31/429; A61K 31/4439; A61K 31/4184; A61K 31/422; A61P 31/00; A61P 31/04; C12Q 1/18; C12Q 1/34; C40B 30/06; G01N 21/64; C07D 241/24

(52) U.S. Cl. 514/224.5; 544/406; 544/32; 544/345; 548/371.4; 548/151; 546/270.4; 548/306.1; 548/228; 514/255.06; 514/250; 514/407; 514/366; 514/339; 514/387; 514/376; 435/32; 435/18; 506/10; 435/7.1

(57) ABSTRACT

The disclosure relates to compounds and methods of inhibiting type three secretion system effector molecules, to methods of detecting compounds that inhibit Yops translocation, and to methods of treating or preventing infections by administering compounds described herein to a subject in need thereof.

### RELATED APPLICATIONS

This application claims the benefit of U.S. provisional application No. 61/350,990, filed Jun. 3, 2010; and U.S. provisional application No. 61/377,458, filed Aug. 26, 2010, the entire contents of each of which are herein incorporated by reference.

### GOVERNMENT SUPPORT

The research leading to the present disclosure was supported in part, by National Institutes of Health (NIH) Grant Nos. AI056058, AI073759, NS053740, T32AI007422, R25GM066567, and DK075720. The U.S. Government may have certain rights in this disclosure.

### BACKGROUND

Yersinia spp. are a family of bacteria that primarily causes disease in animals; humans occasionally get infected zoonotically, most often through the food-borne route. In animals, Y. pseudotuberculosis can cause tuberculosis-like symptoms including localized tissue necrosis and granulomas in the spleen, liver and lymph node. In humans, symptoms include fever and right-sided abdominal pain. Y. pseudotuberculosis (Yptb) infections can mimic appendicitis, especially in children and younger adults, and in rare cases the disease can cause skin complaints (erythema nodosum), joint stiffness and pain (reactive arthritis) or spread of bacteria to the blood (bacteremia).

Bacterial type three secretions systems (TTSS) include virulence factors found in many pathogenic gram-negative species, including the pathogenic Yersinia spp. Yptb, for example, requires the translocation of a group of effector molecules, called Yops, to subvert the innate immune response and establish infection. Polarized transfer of Yops from bacteria to immune cells depends on several factors, including the presence of a functional TTSS, the successful attachment of Yersinia to the target cell, and translocon insertion into the target cell membrane.

Since the TTSS is essential for virulence of Yersiniae and other gram-negative pathogens, this system has been a target for development of therapeutics. Screens have been designed to identify inhibitors of TTSS synthesis and/or Yop secretion from bacteria, however no screens have been performed to identify compounds and therapeutic targets that affect Yops translocation (virulence) without dramatically altering the extracellular bacterial structure or reducing bacterial viability. The latter is significant as treating subjects with therapeutic agents that target extracellular components leads to resistant bacterial strains. Due to the pathogenic nature of bacterial infections, and due to the lack of effective therapeutic compounds, there is a need to identify compounds that inhibit translocation of Yops.

### SUMMARY

The present disclosure is based on the discovery that particular compounds inhibit polar translocation of bacterial effectors into host cells, thereby reducing infectivity of the bacteria.

In one embodiment, the disclosure is directed to a method of treating or preventing an infection comprising administering to a subject at risk for, diagnosed with, or exhibiting symptoms of an infection a compound that inhibits Yop translocation. In a particular embodiment, the compound is selected from the group consisting of: N-(4-ethoxyphenyl)pyrazine-2-carboxamide, (C7); 4-phenyl-1,4-dihydroindeno[1,2-d][1,3]thiazine-2,5-dione, (C15); furo[3,2-b]quinoxalin-3-yl-(4-phenylpiperazin-1-yl)methanone, (C19); 1-[(E)-(3,5-dimethyl-1-phenyl-pyrazol-4-yl)iminomethyl]naphthalen-2-ol, (C20); (2Z)-2-[(3-chloro-5-ethoxy-4-hydroxy-phenyl)methylene]-5,6-dimethyl-thiazolo[3,2-a]benzimidazol-1-one, (C22); 4-(1H-indol-3-yl)-2-(4-pyridyl)thiazole, (C24); 1-(1,3-dimethyl-2-oxo-6-pyrrolidin-1-yl-benzimidazol-5-yl)-3-(3,4-dimethylphenyl)urea, (C34); and (4E)-4-[(2,3-dihydro-1,4-benzodioxin-6-ylamino)methylene]-2-(p-tolyl)oxazol-5-one (C38) and salts, and derivatives, and substituted structures thereof. C7, C15, C19, C20, C22, C24, C34 and C38, and salts, derivatives and substituted structures thereof. In certain embodiments, the compounds are substituted with one or more substituent. In a particular embodiment, the infection is from a pathogen comprising a TTSS, e.g., a gram negative bacterium, e.g., a Yersinia bacterium. In a particular embodiment, the compound inhibits Yop translocation without affecting synthesis of TTSS components. In a particular embodiment, the subject is a mammal, e.g., a human.

In certain embodiments, the compounds are administered in combination with a second antibacterial agent.

One embodiment of the present disclosure is directed to a pharmaceutical composition comprising a compound is selected from the group consisting of: C7, C15, C19, C20, C22, C24, C34 and C38, and salts, derivatives and substituted structures thereof. In a particular embodiment, the pharmaceutical composition further comprises a second anti-bacterial agent.

One embodiment of the present disclosure is directed to a method of treating or preventing a bacterial infection comprising administering to a subject at risk for, diagnosed with, or exhibiting symptoms of a bacterial infection a pharmaceutical composition of the present disclosure. In a particular embodiment, the subject is diagnosed with a gram negative bacterial infection. In a particular embodiment, the subject is a mammal, e.g., a human.

One embodiment is directed to a method of identifying anti-bacterial activity of a compound comprising: a) mixing a sample comprising a test compound, a Yop, and a cell; and b) measuring inhibition of Yop translocation into the cell, wherein a statistically relevant inhibition of Yop translocation is indicative of the anti-bacterial activity of the compound.

One embodiment of the present disclosure is directed to a method of identifying a compound that inhibits Yop translocation into a cell comprising: a) incubating a recombinant bacterial strain that expresses a chimeric protein comprising a Yop sequence and exogenous sequence, and a cell comprising a detectably labeled reporter, wherein the reporter alters its signal in the presence of the exogenous sequence of the chimeric protein, in the presence or absence of a test compound under conditions that allow for translocation of the chimeric protein into the cell in the absence of a test compound; and b) detecting the reporter to determine if it is in the presence of the chimeric protein; wherein if the reporter is not in the presence of the chimeric protein, then the test compound inhibits Yop translocation. In a particular embodiment, the reporter is fluorescently labeled. In a particular embodiment, the reporter comprises two is fluorescent labels that form a fluorescent resonance energy transfer pair, wherein the first fluorescent label emits energy at the excitation wavelength of the second fluorescent label. In a particular embodiment, the two fluorescent labels are connected by a lactam ring. In a particular embodiment, the two fluorescent labels are coumarin and CCF2. In a particular embodiment, the Yop sequence is the N-terminal 100 amino acids of YopE. In a particular embodiment, the exogenous sequence of the chimeric protein comprises an enzymatic activity that modifies the reporter. In a particular embodiment, the enzymatic activity is a lactamase activity.

One embodiment of the present disclosure is directed to a kit comprising a pharmaceutical composition comprising a compound is selected from the group consisting of: C7, C15, C19, C20, C22, C24, C34 and C38, and salts, derivatives and substituted structures thereof, and one or more reagents for administering the pharmaceutical composition to a subject.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A and B show results and the strategy for the high throughput screen (HTS) for small molecule inhibitors of Yop translocation and fluorescence micrographs. FIG. 1A shows fluorescence micrographs of HEp-2 cells loaded with CCF2-AM. From left to right: uninfected cells, HEp-2 cells infected with WT E-TEM, and ΔyopB E-TEM. FIG. 1B is a schematic representation of the HTS (see Example).

FIGS. 2A and B are images showing cell-rounding assays. YopE mediated cell-rounding was reduced after exposure of bacteria and HEP-2 cells to compounds. FIG. 2A shows micrographs of the cell rounding assay described in the Example. Conditions were as indicated in the panels. FIG. 2B fluorescence micrographs of FITC-conjugated rhodamine (phalloidin) treated cells to visualize the actin cytoskeleton. Test compounds are as indicated.

FIGS. 3A and B are graphs showing the toxicity effects of the test compounds, and FIG. 3C shows the structures of the test compounds. FIG. 3A is a graph showing nine of the compounds had no effect on bacterial growth under experimental conditions (DMSO (▪); C7 (▴); C15 (▾); C19 (♦); C20 (); C22 (□); C24 (Δ); C34 (∇); C38 (⋄)). FIG. 3B shows LDH release from HEp-2 cells in the presence of 60 μM compounds. The amount of LDH released into the supernatants was determined at 2 hours and 24 hours. The means and standard deviations from one representative experiment are plotted. FIG. 3C shows the structures of compounds that inhibited cell-rounding of HEp-2 cells at a concentration of 60 μM, but were not bactericidal or cytotoxic in HEp-2 cells.

FIGS. 4A-C show data indicating the localization and assembly of extracellular YscF and LcrV after exposure to compounds. FIG. 4A depicts micrographs showing localized YscF antibody (showing up as dots surrounding the cell). FIG. 4B shows the results of chemical crosslinking to determine whether the compounds affected the TTSS needle structure (see Example). FIG. 4C shows the effect of compounds on Yops secretion. Cultures of Yptb were grown in secretion media and 60 μM compounds. Yop secretion was detected by precipitation of cultured supernatants in 10% trichloroacetic acid (TCA). Proteins were separated by SDS-PAGE and stained with coomassie blue to detect secreted Yops. Protein concentration was normalized to OD and equivalent amounts loaded in each lane.

FIGS. 5A-C are graphs, and corresponding gel images, showing translocation of YopE into HEp-2 cells reveals a translocation defect caused by compounds. FIG. 5A shows the extent of YopE translocation. FIG. 5B shows the extent of YopE synthesis. FIG. 5C shows the extent of YopE leaked into the media. Asterisks (*) indicate p≦0.05. Double asterisk indicates p≦0.06.

FIGS. 6A-E are graphs showing adherence of Yptb to HEp-2 cells as measured by ELISA (see Example). FIG. 6A shows adherence of Yptb to HEp-2 cells in the presence of test compounds. FIG. 6B shows adherence of WT and adherence-defective Yersinia strains. FIG. 6C shows agglutination of WT and yadA defective Yersinia. FIG. 6D shows adherence to E. coli strains. FIG. 6E shows the extent of hemolysis in the presence of DMSO and C20. The means and standard deviations shown from one representative experiment are shown.

FIG. 7 show micrographs of P. aeruginosa ExoS-dependent cell-rounding, demonstrating that cell-rounding was blocked by C20, C22, C24, C34, and C38. The experiment was repeated twice and representative micrographs are shown.

FIG. 8 is a schematic diagram showing the summary of results for compounds identified in the screen for small molecule inhibitor of Yop translocation.

### DETAILED DESCRIPTION

Described herein are screens useful for identifying small molecules that block translocation of bacterial effectors (e.g., Yops) into mammalian cells. The small molecules that were identified are unique in that they permit secretion of Yops from bacteria, but they reduce the polarized translocation of Yops into target cells and cause excessive leakage of Yops into culture supernatants. These compounds represent novel agents that target effector translocation, an essential process for virulence in Yersiniae and other TTSS-containing pathogens.

A high-throughput screen was employed to identify small molecules that block translocation of Yops into mammalian cells. Six compounds were identified that inhibited translocation of effectors without affecting synthesis of TTSS components and secreted effectors, assembly of the TTSS, or secretion of effectors. One compound, C20, reduced adherence of Yptb to target cells. The compounds additionally caused leakage of Yops into the supernatant during infection and thus reduced polarized translocation. Several molecules, C20, C22, C24, C34 and C38, also inhibited ExoS-mediated cell-rounding, suggesting that the compounds target conserved factors between P. aeruginosa and Yptb.

Many pathogenic gram-negative bacteria encode a type three secretion system (TTSS) that translocates effector proteins into the cytosol of their eukaryotic cell targets. Once introduced into host cells, these proteins subvert normal cell functions, such as disrupting innate immune signaling or modulating the phagosomal environment (Black, D. et al., 2000. Mol. Microbiol., 37:515-27; Monack, D. et al., 1997. Proc. Natl. Acad. Sci. USA, 94:10385-90; Rosqvist, R. et al., 1991. Infect. Immun., 59:4562-9; Trosky, J. et al., 2008. Cell Microbiol., 10:557-65). TTSSs are comprised of a base structure, a needle and a tip/translocon complex (Mueller, C. et al., 2008. Mol. Microbiol., 68:1085-95). The base structure, which spans the inner and outer membrane shares high structural homology to conserved bacterial flagellar machinery (Blocker, A. et al., 2003. Proc. Natl. Acad. Sci. USA, 100:3027-30). High-resolution microscopy of the base structures of Shigella and Salmonella reveal that the base consists of several ring structures that surround a hollow cavity (Blocker, A. et al., 2001. Mol. Microbiol., 39:652-63; Kubori, T. et al., 2000. Proc. Natl. Acad. Sci. USA, 97:10225-30; Marlovits, T. et al., 2004. Science, 306:1040-2). The needle is comprised of a small protein that polymerizes to form a hollow tube that starts within the base and protrudes from the bacterial surface (Hoiczyk, E. & G. Blobel, 2001. Proc. Natl. Acad. Sci. USA, 98:4669-74; Tamano, K. et al., 2000. EMBO J., 19:3876-87). Effectors are thought to be translocated through the needle (Davis, A. & J. Mecsas, 2007. J. Bacteriol., 189:83-97; Jin, Q. & S. He, 2001. Science, 294:2556-8), although this has not been conclusively demonstrated for many systems. Many TTSS secrete effectors into culture supernatants with just the base and needle; however, translocation of effectors into mammalian cells requires three additional components, called the translocon (Hakansson, S. et al., 1996. EMBO J., 15:5812-23, Holmstrom, A. et al., 2001. Mol. Microbiol., 39:620-32). Two proteins (Blocker, A. et al., 1999. J. Cell Biol., 147:683-93, Neyt, C. & G. Cornelis, 1999. Mol. Microbiol., 33:971-81), are inserted into the eukaryotic cell membrane to form a pore. The third (Mueller, C. et al., 2005. Science, 310:674-6) is critical for proper assembly of the translocon and is localized at the distal end of the needle, but is not inserted into the host plasma membrane.

There are at least three species of Yersinia that are pathogenic to humans. Y. pseudotuberculosis (Yptb) (Huang, X. et al., 2006. Clin. Med. Res., 4:189-99; Fisher, M. et al., 2007. Infect. Immun., 75:429-42) and Y. enterocolitica both cause gastroenteritis and lymphadenitis and are commonly transmitted via the fecal-oral route (Putzker, M. et al., 2001. Clin. Lab., 47:453-66). Y. pestis is the causative agent of bubonic and pneumonic plague and is commonly transmitted by a flea vector from infected rodents to humans (Achtman, M. et al., 1999. Proc. Natl. Acad. Sci. USA, 96:14043-8; Brubaker, R., 1991. Clin. Microbiol. Rev., 4:309-24). It disseminates through the skin to the lymph nodes where it causes a bubonic disease. Occasionally, Y. pestis disseminates to the lungs of the infected individual, which can lead to a pneumonic transmission from person to person resulting in a fatal lung infection (Lathem, W. et al., 2005. Proc. Natl. Acad. Sci. USA, 102:17786-91). The TTSS is an essential virulence factor for all three pathogenic Yersinia spp (Cornelis, G., 2002. Nat. Rev. Mol. Cell Biol., 3:742-52; Mulder, B. et al., 1989. Infect. Immun., 57:2534-41). Yersinia strains lacking this secretion system can function as live-attenuated vaccine strains in mice (Okan, N. et al., 2010. Infect. Immun., 78:1284-93). The critical needle and translocation components of the Yersinia TTSS include the needle protein, YscF, the tip protein, LcrV, and the pore-forming proteins, YopB and YopD (Marenne, M. et al., 2003. Microb. Pathog., 35:243-58; Tardy, F. et al., 1999. EMBO J., 18:6793-9). The effector proteins translocated by the Yersinia TTSS, called Yops, are targeted to neutrophils, macrophages and dendritic cells where they inactivate the bactericidal effects of these cells during murine infection (Koberle, M. et al., 2009. PLoS Pathog., 5:e1000551; Marketon, M. et al., 2005. Science, 309:1739-41). Inactivation of the TTSS leads to defective colonization of systemic organs and clearance of the bacteria by the host organism (Balada-Llasat, J. et al., 2007. Vaccine, 25:1526-33; Hartland, E. et al., 1996. Infect. Immun., 64:2308-14; Une, T. & R. Brubaker, 1984. Infect. Immun., 43:895-900).

The process of translocation in Yersinia requires close contact between the host cell and the bacterium (Bliska, J. et al., 1993. Infect. Immun., 61:3914-21). In the enteric Yersinia spp., this contact is mediated by two adhesins, YadA and Invasin (Isberg, R. et al., 1987. Cell, 50:769-78; Young, V. et al., 1990. Mol. Microbiol., 4:1119-28). Both of these molecules bind β1 integrins on the surface of target cells (Eitel, J. & P. Dersch, 2002. Infect. Immun., 70:4880-91; Isberg, R. & J. Leong, 1990. Cell, 60:861-71). In cultured cells, stimulation of β1-integrins by ligands activates Src kinases and RhoA, which, in turn, enhances translocation of Yops (Mejia, E. et al., 2008. PLoS Pathog., 4:e3). In the absence of Yops, activation of β1 integrins leads to actin rearrangements resulting in bacterial internalization (Mohammadi, S. & R. Isberg, 2009. Infect. Immun., 77:4771-82). However, in Yersinia expressing the TTSS and Yops this process is antagonized by the effector proteins. The end result is that virulent Yersinia adheres tightly to mammalian cells while remaining extracellular.

One embodiment of the disclosure is directed to a screen for identifying agent(s) from a set of test agents, wherein the identified agent(s) inhibit polar translocation of Yops without affecting other life cycle or structural elements of TTSS-containing bacteria. Screen(s) described herein take advantage of a reporter system that is indicative of translocation of Yops from bacteria to a host cell. As used herein, “reporter” refers to a molecule or signal that is indicative of a particular state. For example, as noted in the Example, where a cleavable molecule is present, it acts as a reporter to indicate whether it is in the presence of a cleaving agent, as it signals in a particular manner in its uncleaved state and in a different manner in its cleaved state.

The cleavable reporter described in the Example utilizes a lactam ring to link two fluorescent labels that form a FRET pair. As used herein, a “FRET pair” refers to a pair of fluorescent labels wherein when the first label is excited at its excitation wavelength, it emits at the excitation wavelength of the second label, thereby causing, if the two labels are in close proximity, the second label to emit at its emission wavelength. Thus, a FRET pair, when excited at the excitation wavelength of the first label, emits at the emission wavelength of the second label. A FRET pair, e.g., coumarin and CCF2, linked via a lactam ring can be cleaved by a suitable lactamase. Described herein, for example, is an active lactamase fragment joined to a YopE fragment, such that the YopE fragment contains the translocation signal required to normally translocate YopE into a host cell. If a host cell containing the FRET pair joined by the lactam ring is infected with a bacterium carrying the Yop/Lac fusion protein, then the lactam ring will be cleaved and the two labels will be separated. In such a scenario, excitation at the excitation wavelength of the first label will cause emission at the emission wavelength of the first label—not the second. The reporter, therefore, is indicative of whether the Yop/Lac fusion protein is translocated into the host cell, as it will emit light at the emission wavelength of the first label if the Yop/Lac fusion is present, and it will emit light at the emission wavelength of the second label if the Yop/Lac fusion protein is not present.

As used herein, “fragment” refers to a portion of a molecule, e.g., a gene, coding sequence, or protein, that has a desired length or function. A “YopE” fragment, for example, can be a fragment of the full length YopE protein that contains the protein signal required for translocation into a host cell. A Lac fragment, for example, can be the portion of the β-lactamase gene that retains lactamase activity. In general, a fragment of an enzyme or signaling molecule can be, for example, that portion(s) of the molecule that retains its signaling or enzymatic activity. A fragment of a gene or coding sequence, for example, can be that portion of the gene or coding sequence that produces an expression product fragment. As used herein, “gene” is a term used to describe a genetic element that gives rise to expression products (e.g., pre-mRNA, mRNA, and polypeptides). A fragment does not necessarily have to be defined functionally, as it can also refer to a portion of a molecule that is not the whole molecule, but has some desired characteristic or length (e.g., restriction fragments, amplification fragments, etc.).

The present disclosure provides, for example, for the use of chimeric proteins encoded by particular gene fragments. One of skill in the art will recognize that conservative substitutions can be made to gene sequences that result in functional expression products. As such, the gene fragments used herein to create chimeric proteins can be wild-type sequences of desired proteins, or they can be variant sequences that share a high homology with wild-type sequences. The disclosure provides for the use of sequences that at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to desired wild-type sequences. As used herein, the term “about” means plus or minus 10% of the numerical value of the number with which it is being used. About 50%, for example, means in the range of 45%-55%. The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art.

In other aspects, the disclosure also provides vectors (e.g., plasmid, phage, expression), cell lines (e.g., mammalian, insect, yeast, bacterial), and kits comprising any of the sequences of the disclosure described herein.

One embodiment of the disclosure is directed to using one or more of the identified agents identified herein or identified through the use of a screen described herein to treat a bacterial infection, e.g., wherein the pathogen is, for example, a gram-negative bacterium, bacteria that utilizes a TTSS, etc. The compounds identified herein or identified through the screens described herein can be delivered in a variety of formulations and amounts to achieve desired effects.

“Treatment” refers to the administration of medicine or the performance of medical procedures with respect to a patient or subject, for either prophylaxis (prevention) or to cure the infirmity or malady in the instance where the patient is afflicted. Prevention of infection is included within the scope of treatment. The compounds described herein or identified through methods described herein can be used as part of a treatment regimen in therapeutically effective amounts. A “therapeutically effective amount” is an amount sufficient to decrease, prevent or ameliorate the symptoms associated with a medical condition. e.g., bacterial infection or symptoms related to bacterial infection. The present disclosure, for example, is directed to treatment using a therapeutically effective amount of a compound sufficient to prevent infection or to reduce virulence of a bacterial strain after infection.

The terms “patient” and “subject” mean all animals including humans. Examples of patients or subjects include humans, cows, dogs, cats, rabbits, goats, sheep and pigs.

The treatment(s) described herein are understood to utilize formulations including compounds identified herein or identified through methods described herein and, for example, salts, solvates and co-crystals of the compound(s). The compounds of the present disclosure can exist in unsolvated as well as solvated forms with pharmaceutically acceptable solvents such as, for example, water, ethanol, and the like. In general, the solvated forms are considered equivalent to the unsolvated forms for the purposes of the present disclosure.

The term “pharmaceutically acceptable salts, esters, amides, and prodrugs” as used herein refers to those carboxylate salts, amino acid addition salts, esters, amides, prodrugs and inclusion complexes of the compounds of the present disclosure that are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The term “prodrug” refers to compounds that are rapidly transformed in vivo to yield the parent compounds of the above formula, for example, by hydrolysis in blood (T. Higuchi and V. Stella, “Pro-drugs as Novel Delivery Systems,” Vol. 14 of the A.C.S. Symposium Series; Bioreversible Carriers in Drug Design, ed. Edward B. Roche, American Pharmaceutical Association and Pergamon Press, 1987; both of which are incorporated herein by reference in their entirety). Activation in vivo may come about by chemical action or through the intermediacy of enzymes. Microflora in the GI tract may also contribute to activation in vivo.

The term “solvate” refers to a compound in the solid state, wherein molecules of a suitable solvent are incorporated. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Co-crystals are combinations of two or more distinct molecules arranged to create a unique crystal form whose physical properties are different from those of its pure constituents (Remenar, J. et al., 2003. J. Am. Chem. Soc., 125:8456-8457) and fluoxetine. Inclusion complexes are described in Remington: The Science and Practice of Pharmacy 19.sup.th Ed. (1995) volume 1, page 176-177. The most commonly employed inclusion complexes are those with cyclodextrins, and all cyclodextrin complexes, natural and synthetic, with or without added additives and polymer(s), as described in U.S. Pat. Nos. 5,324,718 and 5,472,954. The disclosures of Remenar, Remington and the '718 and '954 patents are incorporated herein by reference.

The compounds may be presented as salts. The term “pharmaceutically acceptable salt” refers to salts whose counter ion derives from pharmaceutically acceptable non-toxic acids and bases. Suitable pharmaceutically acceptable base addition salts for the compounds of the present disclosure include, but are not limited to, metallic salts made from aluminum, calcium, lithium, magnesium, potassium, sodium and zinc or organic salts made from lysine, N,N-dialkyl amino acid derivatives (e.g. N,N-dimethylglycine, piperidine-1-acetic acid and morpholine-4-acetic acid), N,N′-dibenzylethylenediamine, chloroprocaine, choline, diethanolamine, ethylenediamine, meglumine (N-methylglucamine) and procaine. Where the compounds contain a basic residue, suitable pharmaceutically acceptable base addition salts for the compounds of the present disclosure include, for example, inorganic acids and organic acids. Examples include acetate, benzenesulfonate (besylate), benzoate, bicarbonate, bisulfate, carbonate, camphorsulfonate, citrate, ethanesulfonate, fumarate, gluconate, glutamate, bromide, chloride, isethionate, lactate, maleate, malate, mandelate, methanesulfonate, mucate, nitrate, pamoate, pantothenate, phosphate, succinate, sulfate, tartrate, p-toluenesulfonate, and the like (Barge, S et al., 1977. J. Pharm. Sci., 66:1-19, the contents of which are incorporated herein by reference).

Diluents that are suitable for use in the pharmaceutical composition of the present disclosure include, for example, pharmaceutically acceptable inert fillers such as microcrystalline cellulose, lactose, sucrose, fructose, glucose dextrose, or other sugars, dibasic calcium phosphate, calcium sulfate, cellulose, ethylcellulose, cellulose derivatives, kaolin, mannitol, lactitol, maltitol, xylitol, sorbitol, or other sugar alcohols, dry starch, saccharides, dextrin, maltodextrin or other polysaccharides, inositol or mixtures thereof. The diluent can be, for example, a water-soluble diluent. Examples of preferred diluents include, for example: microcrystalline cellulose such as Avicel PH112, Avicel PH101 and Avicel PH102 available from FMC Corporation; lactose such as lactose monohydrate, lactose anhydrous, and Pharmatose DCL 21; dibasic calcium phosphate such as Emcompress; mannitol; starch; sorbitol; sucrose; and glucose. Diluents are carefully selected to match the specific composition with attention paid to the compression properties. The diluent can be used in an amount of about 2% to about 80% by weight, about 20% to about 50% by weight, or about 25% by weight of the treatment formulation.

Other agents that can be used in the treatment formulation include, for example, a surfactant, dissolution agent and/or other solubilizing material. Surfactants that are suitable for use in the pharmaceutical composition of the present disclosure include, for example, sodium lauryl sulphate, polyethylene stearates, polyethylene sorbitan fatty acid esters, polyoxyethylene castor oil derivatives, polyoxyethylene alkyl ethers, benzyl benzoate, cetrimide, cetyl alcohol, docusate sodium, glyceryl monooleate, glyceryl monostearate, glyceryl palmitostearate, lecithin, medium chain triglycerides, monoethanolamine, oleic acid, poloxamers, polyvinyl alcohol and sorbitan fatty acid esters. Dissolution agents increase the dissolution rate of the active agent and function by increasing the solubility of the active agent. Suitable dissolution agents include, for example, organic acids such as citric acid, fumaric acid, tartaric acid, succinic acid, ascorbic acid, acetic acid, malic acid, glutaric acid and adipic acid, which may be used alone or in combination. These agents can also be combined with salts of the acids, e.g., sodium citrate with citric acid, to produce a buffer system. Other agents that can be used to alter the pH of the microenvironment on dissolution include salts of inorganic acids and magnesium hydroxide.

Disintegrants that are suitable for use in the pharmaceutical composition of the present disclosure include, for example, starches, sodium starch glycolate, crospovidone, croscarmellose, microcrystalline cellulose, low substituted hydroxypropyl cellulose, pectins, potassium methacrylate-divinylbenzene copolymer, poly(vinyl alcohol), thylamide, sodium bicarbonate, sodium carbonate, starch derivatives, dextrin, beta cyclodextrin, dextrin derivatives, magnesium oxide, clays, bentonite and mixtures thereof.

The active ingredient of the present disclosure can be mixed with excipients, which are pharmaceutically acceptable and compatible with the active ingredient and in amounts suitable for use in the therapeutic methods described herein. Various excipients can be homogeneously mixed with the active agent of the present disclosure as would be known to those skilled in the art. The active agent, for example, can be mixed or combined with excipients such as but not limited to microcrystalline cellulose, colloidal silicon dioxide, lactose, starch, sorbitol, cyclodextrin and combinations of these.

Compositions of the present disclosure may also optionally include other therapeutic ingredients, anti-caking agents, preservatives, sweetening agents, colorants, flavors, desiccants, plasticizers, dyes, and the like.

In certain embodiments, the compositions are administered in combination with a second antibacterial agent such as, for example, Streptomycin, Neomycin (Framycetin, Paromomycin, Ribostamycin), Kanamycin (Amikacin, Arbekacin, Bekanamycin, Dibekacin, Tobramycin), Spectinomycin, Hygromycin B, Paromomycin, Gentamicin (Netilmicin, Sisomicin, Isepamicin), Verdamicin, Astromicin, Doxycycline, Chlortetracycline, Clomocycline, Demeclocycline, Lymecycline, Meclocycline, Metacycline, Minocycline, Oxytetracycline, Penimepicycline, Rolitetracycline, Tetracycline, Tigecycline, Oxazolidinone, Linezolid, Torezolid, Eperezolid, Posizolid, Radezolid, Chloramphenicol, Azidamfenicol, Thiamphenicol, Florfenicol, Retapamulin, Tiamulin, Valnemulin, Erythromycin, Azithromycin, Spiramycin, Midecamycin, Oleandomycin, Roxithromycin, Josamycin, Troleandomycin, Clarithromycin, Miocamycin, Rokitamycin, Dirithromycin, Flurithromycin, Ketolide (Telithromycin, Cethromycin, Solithromycin), Clindamycin, Lincomycin, Pristinamycin, Quinupristin/dalfopristin, Virginiamycin (Fosfomycin), DADAL/AR inhibitors (Cycloserine), bactoprenol inhibitors (Bacitracin), Vancomycin (Oritavancin, Telavancin), Teicoplanin (Dalbavancin), Ramoplanin, Amoxicillin, Ampicillin (Pivampicillin, Hetacillin, Bacampicillin, Metampicillin, Talampicillin), Epicillin, Carbenicillin (Carindacillin), Ticarcillin, Temocillin, Azlocillin, Piperacillin, Mezlocillin, Mecillinam (Pivmecillinam), Sulbenicillin, Clometocillin, Benzathine benzylpenicillin, Procaine benzylpenicillin, Azidocillin, Penamecillin, Phenoxymethylpenicillin (V), Propicillin, Benzathine phenoxymethylpenicillin, Pheneticillin, Cloxacillin (Dicloxacillin, Flucloxacillin), Oxacillin, Meticillin, Nafcillin, Faropene, Biapenem, Ertapenem, antipseudomonal (Doripenem, Imipenem, Meropenem), Panipenem, Cefazolin, Cefacetrile, Cefadroxil, Cefalexin, Cefaloglycin, Cefalonium, Cefaloridine, Cefalotin, Cefapirin, Cefatrizine, Cefazedone, Cefazaflur, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, cephamycin (Cefoxitin, Cefotetan, Cefinetazole), carbacephem (Loracarbef), Cefixime, Ceftriaxone, (Ceftazidime, Cefoperazone), Cefcapene, Cefdaloxime, Cefdinir, Cefditoren, Cefetamet, Cefinenoxime, Cefodizime, Cefotaxime, Cefpimizole, Cefpiramide, Cefpodoxime, Cefsulodin, Cefteram, Ceftibuten, Ceftiolene, Ceftizoxime, oxacephem (Flomoxef, Latamoxef), Cefepime, Cefozopran, Cefpirome, Cefquinome, Ceftobiprole, Ceftaroline fosamil, Ceftiofur, Cefquinome, Cefovecin, Aztreonam, Tigemonam, Carumonam, Tabtoxin, penam (Sulbactam, Tazobactam), clavam (Clavulanic acid), Co-amoxiclav (Amoxicillin/clavulanic acid), Imipenem/cilastatin, Ampicillin/sulbactam (Sultamicillin), Piperacillin/tazobactam, Sulfaisodimidine, Sulfamethizole, Sulfadimidine, Sulfapyridine, Sulfafurazole, Sulfanilamide (Prontosil), Sulfathiazole, Sulfathiourea, Sulfamethoxazole, Sulfadiazine, Sulfamoxole, Sulfadimethoxine, Sulfalene, Sulfametomidine, Sulfametoxydiazine, Sulfamethoxypyridazine, Sulfaperin, Sulfamerazine, Sulfaphenazole, Sulfamazone, sulfanilamide (Sulfacetamide, Sulfametrole), Trimethoprim/sulfamethoxazole, Cinoxacin, Flumequine, Nalidixic acid, Oxolinic acid, Pipemidic acid, Piromidic acid, Rosoxacin, Ciprofloxacin, Enoxacin, Fleroxacin, Lomefloxacin, Nadifloxacin, Ofloxacin, Norfloxacin, Pefloxacin, Rufloxacin, Balofloxacin, Grepafloxacin, Levofloxacin, Pazufloxacin, Sparfloxacin, Temafloxacin, Tosufloxacin, Besifloxacin, Clinafloxacin, Garenoxacin, Gemifloxacin, Moxifloxacin, Gatifloxacint, Sitafloxacin, Trovafloxacin/Alatrofloxacin, Prulifloxacin, Danofloxacin, Difloxacin, Enrofloxacin, Ibafloxacin, Marbofloxacin, Orbifloxacin, Pradofloxacin, Sarafloxacin, Novobiocin, Metronidazole, Tinidazole, Ornidazole, Nitrofurantoin, Furazolidone, Nifurtoinol, Rifampicin, Rifabutin, Rifapentine, Rifaximin, Xibornol, Clofoctol, Methenamine, Mandelic acid, Nitroxoline, Mupirocin or combinations thereof.

Any such optional ingredient must, of course, be compatible with the compound of the disclosure to insure the stability of the formulation. The dose range for adult humans is generally from 0.1 μg to 10 g/day orally. Tablets or other forms of presentation provided in discrete units can conveniently contain an amount of compound of the disclosure that is effective at such dosage or as a multiple of the same, for instance, units containing 0.1 mg to 500 mg, usually around 5 mg to 200 mg. The precise amount of compound administered to a patient will be the responsibility of the attendant physician. The dose employed will depend on a number of factors, including, for example, the age and sex of the patient, the precise disorder being treated, and its severity. The frequency of administration will depend on the pharmacodynamics of the individual compound and the formulation of the dosage form, which may be optimized by methods well known in the art (e.g., controlled or extended release tablets, enteric coating etc.).

In certain embodiments, the compounds disclosed herein are optionally substituted with one or more substituents.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context include, for example, halogen, hydroxy, alkyl, alkoxy, alkanoyl, nitro, cyano, oxo, carbocyclyl, carbocycloalkyl, heterocarbocyclyl, heterocarbocycloalkyl, aryl, arylalkyl, heterocarbocyclyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen, hydroxyl, alkyl, alkoxy, alkanoyl, amino, alkylamino, dialkylamino, alkylthiol, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom or compound is unsubstituted.

As used herein, “alkyl” means a noncyclic straight chain or branched, unsaturated or saturated hydrocarbon such as those containing from 1 to 10 carbon atoms, while the term “lower alkyl” or “C1-6alkyl” has the same meaning as alkyl but contains from 1 to 6 carbon atoms. The term “higher alkyl” has the same meaning as alkyl but contains from 7 to 10 carbon atoms. Representative saturated straight chain alkyls include, for example, methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl,” respectively). Representative straight chain and branched alkenyls include, for example, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include, for example, cyclopropyl, cyclobutyl, cyclopentyl, cyclohexyl, and the like; while unsaturated carbocycles include, for example, cyclopentenyl and cyclohexenyl, aryls and the like.

“Heterocarbocycles” or “heterocarbocyclyl” groups are carbocycles that contain from one to four heteroatoms independently selected from, for example, nitrogen, oxygen and sulfur (which may be saturated or unsaturated (but not aromatic)), monocyclic or polycyclic, and wherein the nitrogen and sulfur heteroatoms can be optionally oxidized, and the nitrogen heteroatom can be optionally quaternized. Heterocarbocycles include, for example, morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl.

As used herein, “heteroaryl” refers an aromatic heterocarbocycle having one to four heteroatoms selected from, for example, nitrogen, oxygen and sulfur, and containing at least one carbon atom, including both mono- and polycyclic ring systems. Polycyclic ring systems can, but are not required to, contain one or more non-aromatic rings, as long as one of the rings is aromatic. Representative heteroaryls are, for example, furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes, for example, N-alkylated derivatives such as a 1-methylimidazol-5-yl substituent.

As used herein, “heterocycle” or “heterocyclyl” refers to mono- and polycyclic ring systems having one to four heteroatoms selected from, for example, nitrogen, oxygen and sulfur, and containing at least one carbon atom. The mono- and polycyclic ring systems can be aromatic, non-aromatic or mixtures of aromatic and non-aromatic rings. Heterocycle includes heterocarbocycles, heteroaryls, and the like.

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy.

“Alkylamino” refers an alkyl group as defined above with the indicated number of carbon atoms attached through an amino bridge. An example of an alkylamino is methylamino, (e.g., —NH—CH₃).

“Alkanoyl” refers to an alkyl as defined above with the indicated number of carbon atoms attached through a carbonyl bride (i.e., —(C═O)alkyl).

The compounds of this disclosure can exist in radiolabeled form, i.e., the compounds may contain one or more atoms containing an atomic mass or mass number different from the atomic mass or mass number most commonly found in nature. Radioisotopes of, for example, hydrogen, carbon, phosphorous, fluorine, and chlorine include ²H, ³H, ¹³C, ¹⁴C, ¹⁵N, ³⁵S, ¹⁸F and ³⁶Cl, respectively. Compounds that contain those radioisotopes and/or other radioisotopes of other atoms are within the scope of this disclosure. Radiolabeled compounds of the present disclosure and prodrugs thereof can generally be prepared by methods well known to those skilled in the art.

The compounds described herein may contain asymmetric centers and may thus give rise to enantiomers, diastereomers, and other stereoisomeric forms. Each chiral center may be defined, in terms of absolute stereochemistry, as (R)— or (S)—. The present disclosure is meant to include all such possible isomers, as well as, their racemic and optically pure forms. Optically active (R)- and (S)-isomers may be prepared using chiral synthons or chiral reagents, or resolved using conventional techniques. The prefix “rac” refers to a racemate. The representation of the configuration of any carbon-carbon double bond appearing herein is selected for convenience only, and unless explicitly stated, is not intended to designate a particular configuration. Thus a carbon-carbon double bond depicted arbitrarily as E may be Z, E, or a mixture of the two in any proportion. Likewise, all tautomeric forms are also intended to be included.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association at least one compound of the present disclosure or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of the active ingredient; as a powder (including micronized and nanoparticulate powders) or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The treatments (therapies) described herein can also be part of “combination therapies.” Combination therapy can be achieved by administering two or more agents, each of which is formulated and administered separately, or by administering two or more agents in a single formulation. The second active ingredient can be, for example, a second compound identified herein or through screens described herein, or active ingredients useful for treating, for example, symptoms of bacterial infections or preventing bacterial infections. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within any number of hours of each other or within any number or days or weeks of each other.

The present disclosure is also directed to kits for treating or preventing bacterial infections comprising compound(s) identified herein or compound(s) identified through the screening methods provided herein. The kits of the present disclosure can include, for example, components necessary for delivering a therapeutically effective amount of the active agent, instructions for use and/or devices for delivery of the active agent(s).

### Example

### Strains and Bacterial Culture Conditions

Strains used are listed in Table 1. Yptb strains were cultured in Luria broth (L-broth) at 26 C overnight with aeration. Unless otherwise indicated, strains were diluted 1:40 into 2×YT containing 5 mM CaCl₂, incubated at 26 C for 1.5 hours with aeration, followed by incubation at 37 C for 1.5 hours with aeration to induce synthesis of the TTSS. Compounds were added at 60 μM final concentration at the shift to 37 C. In some experiments, strains were subcultured 1:40 in secretion media (2×YT supplemented with 20 mM sodium oxalate and 20 mM MgCl₂) and grown as above.

TABLE 1
Strains and plasmids

| Strain | Description |
|---|---|
| E. coli | |
| SY327λpir | Conjugation strain |
| Sm10λpir | Conjugation strain |
| Y. pseudotuberculosis | |
| IP2666 plB1 | Wild-type |
| IP2666 E-TEM | AA 1-100 YopE + TEM1 |
| IP2666 ΔyopB | Deletion of yopB (codons 19-346) |
| IP2666 ΔyopB E-TEM | ΔyopB + AA 1-100 YopE + TEM1 |
| IP2666 ΔyscF | Deletion of yscF (codons 2-86) |
| IP2666 ΔlcrV | Deletion of lcrV (codons 19-326) |
| IP2666 ΔyopN | Deletion of yopN (codons 2-287) |
| IP2666 Δinv | Deletion of inv |
| IP2666 Δinv pYV | Δinv; virulence plasmid minus |
| IP2666 ΔyadA | Deletion of yadA |
| IP2666 Δinv ΔyadA | Deletion of inv and yadA |
| P. aeruginosa strains | |
| Pa388 | Wild-type |
| Pa388 pscC | pscC::Tn5Tc |
| Plasmids | |
| pSR47S | Gene replacement vector; Kan^{r} |
| pSR47S-E-TEM | YopE-TEM1; Kan^{r} |
| pDS132 | Single copy vector; Cm^{r} |
| pDS132-YadA | pDS132 expressing YadA; Cm^{r} |
| pCVD442 | Suicide vector; Ap^{r} |
| pCVD442-ΔyadA | Suicide vector for deleting yadA; Ap^{r} |

E. coli and P. aeruginosa were cultured in L-broth at 37 C with aeration overnight. The next morning P. aeruginosa strains were diluted 1:40 in L-broth and grown at 37 C for 2 hours before the addition of 60 μM compounds. Cultures were then incubated an additional 2 hours before infection of HEp-2 cells. Overnight cultures of E. coli were diluted 1:50 into 2×YT and were grown at 37 C. After a 1.5 hour incubation at 37 C compounds were added to 60 μM concentration, and then incubated at 37 C for another 1.5 hours. Chloramphenicol was used in cultures of E. coli at a concentration of 10 μg/mL for maintenance of pDS132 and pDS132-YadA.

To generate the construct encoding the YopE secretion and translocation signals fused to TEM1, the TEM1 fragment was PCR amplified from pBR322 using primers TEM1 F and TEM1R (See Table 2 for list of primers) and cloned into the pGEM-T easy system (Promega). The TEM1 fragment was then digested with NotI and SacI and cloned into pSR47S generating pSR47S-TEM1. The DNA sequence encoding first 100 amino acids of YopE was PCR amplified using primers ETEMF and ETEMR, cloned into pGEM-T Easy then subcloned into the pSR47s-TEM1 with BamHI and NotI, creating pSR47s-E-TEM. The pSR47s-E-TEM plasmid was introduced into SY327λpir. The YopE-TEM fusion (E-TEM) was introduced at the IP2666 yopE locus by allelic exchange. The E-TEM allele was also introduced into the IP2666 yopB strain to generate yopB E-TEM. Strains were tested to demonstrate that E-TEM, YopE and other Yops were secreted normally when grown under secretion inducing conditions.

The yadA gene was deleted in the Δinv background by allelic exchange (Donnenberg, M. & J. Kaper, 1991. Infect. Immun., 59:4310-7). pCVD442-yadAKO (Durand, E. et al., 2010. Cell Microbiol., 12:1064-82) was conjugated into Δinv IP2666, as described previously (Logsdon, L. & J. Mecsas, 2003. Infect. Immun., 71:4595-607).

TABLE 2
Primers

| Primer | Sequence |
|---|---|
| TEM1F | 5′-GAGAGAGCGGCCGCCACCCAGAAACGCTGGTG (SEQ ID NO: 1) |
| TEM1R | 5′-AGACAGAGCTCGCATGCTGAGTAAACTTGGTCTGACAGT (SEQ ID NO: 2) |
| ETEMF | 5′-GGATCCGCATGCGCACTCTCGGCAGACCATC (SEQ ID NO: 3) |
| ETEMR | 5′-GGCGGCCGCTAGGACTTGGCATTTGTG (SEQ ID NO: 4) |

### Tissue Culture

HEp-2 cells were maintained in RPMI 1640 (Cellgro) with 5% fetal bovine serum at 37 C, and 5% CO₂. For all experiments, HEp-2 cells were seeded into either 6-, 24- or 96-well tissue culture treated plates ˜18 hours prior to experimentation at 6×10⁵, 2×10⁵ or 1.5×10⁴ cells per well, respectively. The following standard procedure for HEp-2 cell infections was used for all tissue culture experiments unless otherwise indicated. Yptb used for infection of HEp-2 cells were gently washed in PBS and resuspended in RPMI with 5% FBS containing 60 μM compounds or 0.3% DMSO. This media+bacteria+compounds mixture was then used to replace culture media on HEp-2 cells. Infection was started by centrifuging the bacteria onto the cells at 290×g for 5 minutes at room temperature (RT). Plates were moved to 37 C, 5% CO₂ for the remainder of the infection.

### High-Throughput Screen

HEp-2 cells were seeded into 384-well plates at a density of 1×10⁴ cell/well in a volume of 25 μL. The CCF2-AM reagent was prepared as per the manufacturer's instructions (Invitrogen). 5 μL of the CCF2 mixture was added to each well to yield a final concentration of 1 μg/mL CCF2 per well and plates were incubated at 30 C for 30 minutes. After incubation, compounds were transferred to plates by pin transfer. Yptb IP2666 E-TEM and IP2666 ΔyopB E-TEM were grown in 2×YT supplemented with 5 mM CaCl₂. Yptb were washed in warm PBS, adjusted to an MOI of 80:1, then added to wells containing CCF2-AM and compounds. The plates were incubated at 37 C for 30 minutes to permit exposure of the bacteria to the compounds before centrifuging at 290×g for 5 minutes to initiate the infection. The infection was allowed to proceed for 60 minutes, 100 μg/ml of gentamicin was added to each well and the green (520 nm) and blue fluorescence (447 nm) were determined on an EnVision plate reader (Perkin Elmer, Waltham, Mass.).

For each experiment, a separate control plate was included with wells that contained only HEp-2 cells to control for background fluorescence signals. In addition, 12 positive controls (WT IP2666 E-TEM) and 12 negative controls (IP2666 ΔyopB E-TEM) were included in the last two rows of each plate as plate-specific controls. To determine the value for green and blue fluorescence in each well, first the background green and blue fluorescence was determined by calculating the average green and blue fluorescence in the control plate containing just HEp-2 cells. The background green and blue fluorescence minus one standard deviation was then subtracted from the green and blue fluorescence values measured from each well in each plate containing compounds. A ratio of blue to green fluorescence in each well was determined and the data were sorted by this ratio to identify wells containing compounds that exhibited low ratios (i.e., reduced translocation of E-TEM). For each plate a Z- and Z′ factor was determined (Zhang, J. et al., 1999. J. Biomol. Screen., 4:67-73). The screen was optimized to yield Z and Z′ values of between 0.2 and 0.5. When the screen was optimized to yield Z values of greater than 0.5, no hits were detected after screening 20,000 compounds. Potential hits were selected based the criteria that their ratios fell outside of three standard deviations for the whole plate and their intrinsic green fluorescence value was within the range of those found in cells infected with ΔyopB E-TEM. Z-factors of <0 were occasionally observed, and in those instances of high variability, there was a very high likelihood for false positives. Plates with Z-factors<0, however, were analyzed and some compounds had a ratio within the range of the ΔyopB controls. In those cases, if the adjusted values of blue and green fluorescence fell within the range of the ΔyopB control wells, compounds were included in a preliminary list of potential hits. Of the 100,000 compounds screened, 200 compounds were deemed potential hits based these criteria. Forty-five compounds were tested in a second assay. Libraries were obtained and screened. A number of libraries were screened including ChemDiv2, ChemDiv3, ChemDiv4, Maybridge3, Maybridge4, and Biomol (as described at the website, iccb.med.harvard.edu/screening/compound_libraries/index.htm). Compounds were diluted in DMSO to 20 mM stocks and stored at −20 C.

### Cell Rounding Assay

HEp-2 cells were seeded into a 96-well plate at a density of 1.5×10⁴ cells/well in a volume of 100 μl. Yptb were diluted to 1.5×10⁶ cfu/mL in RPMI 1640 supplemented with 5% FBS and 60 μM compounds or 0.3% DMSO. The cell culture media was replaced with 100 μL media containing Yptb and compounds. After centrifugation, the infection was allowed to proceed for 45 minutes before imaging. Cells were examined on a Nikon Eclipse TE2000-U microscope (Melville, N.Y., United States).

P. aeruginosa cell rounding assays were performed as above. Infections were allowed to proceed for 90 minutes prior to imaging.

### FITC-Phalloidin Staining

HEp-2 cells were seeded into 96-well plates at a density of 5×10³ cells/well in 100 μl RPMI supplemented with 5% FBS. Compounds were diluted to 60 μM in RPMI with 5% FBS. Media on monolayer was replaced with 100 μl of media with compounds and then incubated at 37 C, 5% CO₂ for 2 hours. Cells were fixed in 4% paraformaldehyde for 20 minutes at room temperature (RT). The monolayer was washed 3× with PBS and permeabilized with the addition of 0.5% Triton-X 100 in PBS for 15 minutes at RT. FITC-phalloidin was added at a concentration of 130 nM and allowed to incubate at RT for 30 minutes. After incubation monolayers were washed 3× with PBS, incubated for 1 minute with 1.6 μM DAPI and then washed another 3× in PBS. Images were obtained with a Nikon inverted TE2000-U microscope (Molecular Devices, Sunnyvale, Calif.).

### Bacterial Growth Curves

WT IP2666 was grown overnight at 26 C in L-broth with aeration. The cultures were then diluted 1:50 into 2×YT and allowed to incubate, shaking at 26 C for 1 hour. At this time, defined as t=0, the OD₆₀₀ values were measured and compounds were added to a final concentration of 60 μM. Cultures were returned to 26 C with aeration and OD₆₀₀ measurements were taken each hour for 7 hours.

### LDH Release Assays

HEp-2 cells were seeded into a 96-well plate at a density of 2×10⁴ cells/well in a volume of 100 μL RPMI supplemented with 5% FBS. Compounds were resuspended in RPMI supplemented with 5% FBS to a final concentration of 60 μM. Media was replaced with a volume of 100 μL and cells were incubated at 37 C and 5% CO₂. A 50 μL sample was taken from the wells at 2 hrs and 24 hrs for quantification of LDH. LDH in supernatants was quantified using the Promega Cytotox 96 Non-Radioactive Cytotoxicity Assay Kit. Control samples for lysis in the absence of compounds were incubated for 45 minutes in Lysis Buffer (supplied in the kit) then collected at the same time points. The OD at 492 nm was measured spectrophotometrically on a SpectraMax 5 plate reader (Molecular Devices, Sunnyvale, Calif.). Each experiment was performed in triplicate and repeated twice.

### Indirect Immunofluorescence Microscopy

Yptb strains were grown in the presence of compounds as described above. Immunfluorescence was performed as described previously (Davis, A. et al., 2010. Mol. Microbiol., 76:236-59). Micrographs were taken with a Nikon inverted TE2000-U microscope with a Photometrics CCD camera at 60× magnification using MetaVue software (Molecular Devices, Sunnyvale, Calif.). DAPI and Alexa 594 were visualized using Nikon UV-2E/C and G-2E/C filters respectively. Images were pseudocolored and merged in MetaVue.

### Chemical Cross-Linking

Yptb were grown in 2×YT supplemented with 5 mM CaCl₂ and with 60 μM compounds as described for the immunofluorescence, and subjected to cross-linking with 1 mM BS³ (Aiello, A. et al., 2010. J. Infect. Dis., 201:491-8).

Secretion of Yops into Culture Supernatants

Secretion of Yops into culture supernatant was performed. Briefly, Yptb were grown in secretion inducing media. After 90 minutes at 37 C in the presence of compounds, supernatants were collected and centrifuged to remove bacteria. The clarified supernatants were precipitated in 10% trichloroacetic acid and resuspended in 2×SDS sample buffer. The secreted proteins were separated by SDS-PAGE and visualized by coomassie staining.

### Translocation and Synthesis Assays

HEp-2 cells were seeded into a 6-well plate at 6×10⁵ cells per well. Yptb were diluted to 3×10⁷ cfu/mL in RPMI 1640 supplemented with 5% FBS and 60 μM compounds or 0.3% DMSO to achieve an MOI of 50:1, 1 mL of this mixture was placed on the HEp-2 cells, then centrifuged at 290×g to initiate the infection. The infection was allowed to proceed for 1 hour at 37 C, 5% CO₂. The tissue culture supernatants were collected to determine the amount of YopE leakage. HEp-2 cells were washed 2× with cold PBS and then lysed with eukaryotic lysis buffer (20 mM HEPES pH 7.4, 150 mM NaCl, 0.1% NP-40, 1 μM PMSF, 10 μM leupeptin, 1 μM pepstatin) for 20 minutes at 4 C. Lysates were fractionated by centrifugation and both the soluble fraction (HEp-2 cell cytosol) and insoluble fraction (Yptb, and HEp-2 cell nuclei and membranes) were electrophoresed in a 12.5% tris-glycine polyacrylamide gel. The proteins were transferred to PVDF membrane and probed with α-YopE (1:10,000), α-βactin (1:10,000), or α-S2 (1:10,000) antibody. Secondary antibodies, α-mouse HRP, or α-rabbit HRP were used at 1:10,000. Blots were developed using chemiluminescence (Perkin Elmer Western Lightning per the instruction manual). Images of blots were obtained on a UMax Astra6700 scanner (Techville, Dallas, Tex.) and quantified by densitometry using Image J (National Institutes of Health). YopE detected in the soluble fraction (translocated YopE) was normalized to the amount of S2 protein detected in the insoluble fraction (equivalent to bacterial cell number). This value was then normalized to the amount of Actin protein in the insoluble fraction (equivalent to HEp-2 cell number), to control for sample loading. Percent translocation was determined by comparing the level of YopE detected in the presence of compounds to the DMSO control.

Total bacterial YopE protein was measured by normalizing the amount of YopE detected in the insoluble fraction (containing intact bacteria) to the S2 protein detected in the same fraction. The percent YopE synthesis was determined by comparing the amount of YopE protein detected in the presence of compounds to the DMSO control. The experiment was repeated three times.

### YopE Leakage Determination

Cell culture supernatants collected from the translocation assay described above were centrifuged at 16,000×g for 2 minutes to remove intact Yptb and HEp-2 cells. 800 μL of the clarified supernatants was mixed with YopE antibody then added to protein A beads and an equal volume of TNET buffer (50 mM Tris-HCl, 150 mM NaCl, 5 mM EDTA, pH 8.0, and 1% Triton-X). The samples were incubated rotating at 4 C overnight. Beads were washed 3× in TNET, and then boiled in sample buffer lacking reducing agent to release protein. The samples were run on 12.5% SDS-PAGE, transferred to PVDF and probed for YopE. Clean-Blot HRP reagent (Thermoscientific) was used as the secondary antibody. Proteins were visualized using chemiluminesence. The amount of leaked YopE was normalized to the amount of S2 determined from the insoluble fraction collected in the translocation and synthesis assay described above. The ratio of leaked YopE was arbitrarily set to a value of 1 for the DMSO control, and each infection in the presence of compound compared to it. The experiment was repeated three times.

### Statistical Analysis

Differences in levels of translocation of Yop leakage in the presence of compounds compared to DMSO were determined by a paired T-Test.

### Adherence Assays

HEp-2 cells were seeded into 96-well plates at a density of 2×10⁴ cells/well in a volume of 100 μL RPMI. Yptb were diluted to 2×10⁶ cfu/mL in RPMI containing 60 μM compounds or 0.3% DMSO. 100 μL of the bacteria/compound mixture was added to each well. The bacteria were spun onto the cells at 290×g for 5 minutes, incubated at 37 C for 30 minutes, and then the wells were washed vigorously with ice-cold PBS to remove any unbound Yptb. Both Yptb and HEp-2 cells were fixed in 4% paraformaldehyde for 60 minutes and washed 3× with PBS. An enzyme-linked immunosorbant assay (ELISA) was performed by incubating the wells with a 1:1000 dilution of polyclonal rabbit α-Yersinia antibody in 1% BSA in PBS, at RT for 1.5 hours with gentle shaking, and then cells were washed 3× in PBS and incubated with a 1:10,000 dilution of α-rabbit HRP in 1% BSA in PBS for 1.5 hours with shaking. The HRP activity was visualized with the TMB ELISA reagent (Thermoscientific) and measured spectrophotometrically at OD₄₅₀. The binding of E. coli expressing YadA to HEp-2 cells was determined as above with the modification that a 1:1000 dilution of α-LamB was used to detect E. coli. Each experiment was performed in triplicate and repeated twice. The average and standard error are shown from one experiment.

### YadA Autoagglutination Assay

Yptb were grown at 26 C overnight in L-broth in the presence of 60 μM C20 or 0.3% DMSO. A 200 μL inoculum from the overnight culture was introduced into 2 mL warm RPMI. The cultures were incubated statically for 3 hrs at 37 C, 5% CO₂, in the presence of 60 μM C20 or 0.3% DMSO. The top 100 μL of the culture was removed and the OD₆₀₀ was measured. The cultures were vortexed and the OD₆₀₀ was read again. The ratio of the OD₆₀₀ of the settled culture to the vortexed culture was determined and the ratio of IP2666 in 0.3% DSMO was set to 100%. The ΔyadA in 0.3% DMSO was set to 0% autoagglutination. Each experiment was performed in triplicate and repeated twice. The average and standard error are shown for one experiment.

### Hemolysis of Sheep Red Blood Cells

Yptb were grown in secretion media and supplemented with 60 μM compounds. The Yptb were pelleted at 16,000×g for 2 minutes and then resuspended to a concentration of 1×10⁹ cfu/mL. Sheep red blood cells (SRBCs; Innovative Research, Southfield, Mich.) were washed 3× in cold 1×PBS and resuspended to 1×10⁹ cells/mL in warm RPMI. The SRBC and Yptb were mixed at an MOI of 1:1 in the presence of 60 μM C20 or 0.3% DMSO in a round bottom 96-well plate, and then pelleted at 2,000×g for 7 minutes to bring Yptb into contact with SRBCs. The infection was allowed to proceed for 3 hrs at 37 C and 5% CO₂. After the incubation, RPMI was removed from SRBCs and replaced with cold 1×PBS containing 100 μg/mL gentamicin and 60 μM C20 or 0.3% DMSO. The SRBC were incubated overnight at room temperature. The next morning, the SRBC were gently resuspended, pelleted at 2,000×g and the OD₅₄₅ of the supernatants determined. The percent lysis of SRBCs in the presence of each adhesin mutant or the C20 compound was normalized to the percent lysis of SRBCs by WT Yptb in 0.3% DMSO (set to 100%). The experiment was performed in triplicate and repeated twice.

### Results

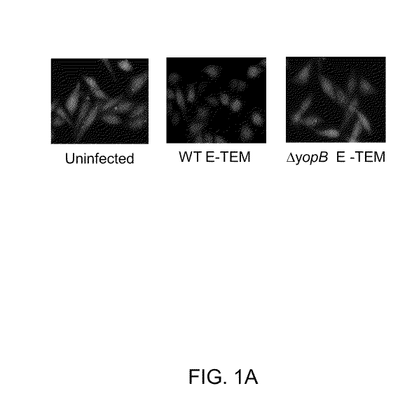

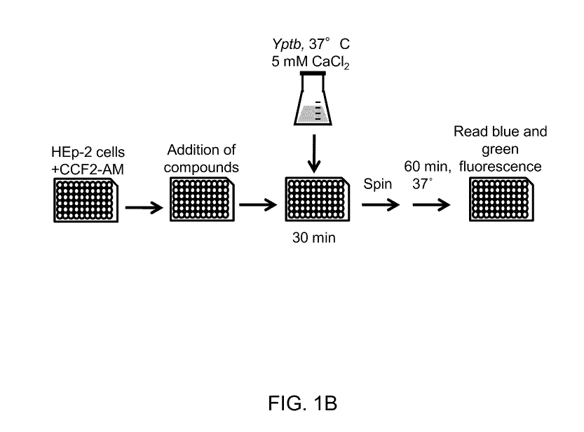

A High-Throughput Screen was designed to identify small molecules that prevent or reduce translocation of Yops into mammalian cells, a critical facet of Yersinia virulence. A fluorescence-based system was used to monitor translocation of a chimeric protein into HEp-2 cells. The chimeric protein, E-TEM, is composed of the first 100 amino acids of YopE, which contains the secretion and translocation signals that are required to direct it into mammalian cells (Sory, M. & G. Cornelis, 1994. Mol. Microbiol., 14:583-94), fused to a fragment of β-lactamase (TEM1) (Charpentier, X. & E. Oswald, 2004. J. Bacteriol., 186:5486-95). A recombinant Yptb strain expressing E-TEM (WT E-TEM) was used to infect HEp-2 cells treated with the membrane-permeable non-fluorescent dye, CCF2-AM (Zlokarnik, G. et al., 1998. Science, 279:84-8). CCF2-AM is comprised of fluorescein conjugated to coumarin by a lactam ring. Outside of the mammalian cell the dye is non-fluorescent but once it is taken up by the cell, it is modified by esterases and becomes trapped. Modified CCF2-AM fluoresces green when excited at 409 nm due to fluorescence resonance energy transfer (FRET) from the coumarin to the fluorescein ring (FIG. 1A, uninfected). If the lactam ring between the fluorophores is cleaved by TEM1, the FRET is lost and the coumarin fluoresces blue (FIG. 1A, WT E-TEM) indicating that E-TEM has been translocated into the host cell. If Yptb are unable to translocate E-TEM into host cells, CCF2-AM will remain uncleaved and the cells will fluoresce green (FIG. 1A, ΔyopB E-TEM). A measure of green to blue conversion can be obtained on a fluorescence plate reader and this assay is therefore amenable to a high-throughput approach.

To screen for small molecules that block translocation, HEp-2 cells were seeded into 384-well plates and incubated with CCF2-AM (FIG. 1B). For each 384-well plate, one row of cells was infected with WT E-TEM but not exposed to compounds, which served as a positive control for translocation. Another row was infected with ΔyopB E-TEM, which secreted Yops and E-TEM, but was unable to translocate them, serving as a negative control. The remaining wells received compounds in concentrations ranging from 20-60 μM as well as WT E-TEM. The Yptb were grown under conditions where the TTSS was expressed and primed for Yop secretion, but Yops were not secreted. WT E-TEM was incubated with the compounds for 30 minutes prior to centrifugation of Yptb onto the monolayer to permit the exposure of Yptb to compounds prior to contact with cells and initiation of infection. Sixty minutes after the initiation of infection, the levels of both green and blue fluorescence in each well was determined by a plate-reader. Raw values for blue and green intensities were adjusted to exclude background fluorescence and the ratios of blue to green fluorescence were calculated. Failure to translocate the E-TEM construct in the presence of compounds resulted in low blue/green fluorescence ratios.

Approximately 100,000 compounds were screened and ranked based on their ratios of blue to green fluorescence. Most wells infected with WT E-TEM and exposed to compounds exhibited ratios that grouped together with the positive controls. Compounds that led to a low blue/green ratio were further analyzed to determine if the low ratio was due to aberrantly high green fluorescence caused by autofluorescence in the wells. Only compounds with low blue/green ratios and green values that fell within the range of the typical observed values for the plate were considered for further analysis.

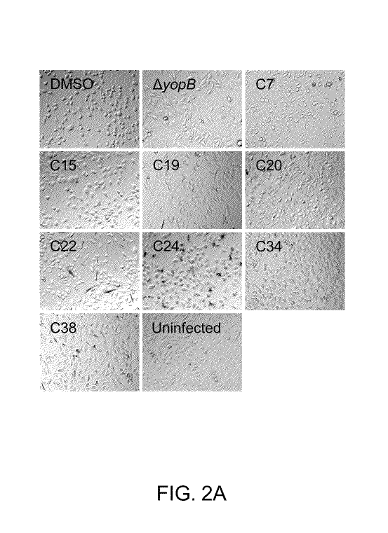

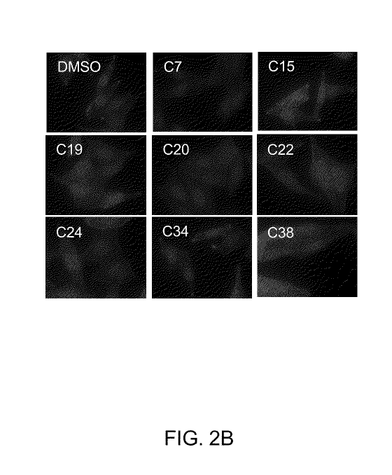

Roughly 200 of the wells exposed to compounds yielded low blue/green ratios. Of these, the top 45 compounds were screened in a second assay that did not rely on TEM1 activity or CCF2-AM fluorescence, but was dependent on translocation of an effector, YopE, into HEp-2 cells. YopE, a Rho-GTPase activating protein (Rho-GAP), disrupts signaling by RhoA, Rac1, and RhoG (Von Pawel-Rammingen, U. et al., 2000. Mol. Microbiol., 36:737-48) leading to rounding of cells and detachment from the tissue culture plate, a phenotype that is easily visualized by light microscopy. The cells infected with WT Yptb in the presence of DMSO alone (FIG. 2A, DMSO) showed a rounded phenotype consistent with high-levels of translocation of YopE. Cells infected with Yptb that are incapable of translocating YopE (FIG. 2A, ΔyopB) remain flat, similar to the uninfected. Of the 45 compounds tested, 13 inhibited cell rounding at 60 μM (FIG. 2A), suggesting that the molecules prevented normal levels of YopE translocation. One of the compounds that diminished cell-rounding could not be obtained in large enough quantities for subsequent characterization.

The compounds were also tested to determine whether they caused perturbations of the actin cytoskeleton of HEp-2 cells, which may influence translocation of Yops into target cells. After a 2 hour exposure to just the compounds, actin stress fibers were detected by FITC-rhodamine. Those compounds that inhibited Yptb-mediated cell-rounding, did not appear to disrupt the actin cytoskeleton of HEp-2 cells (FIG. 2B).

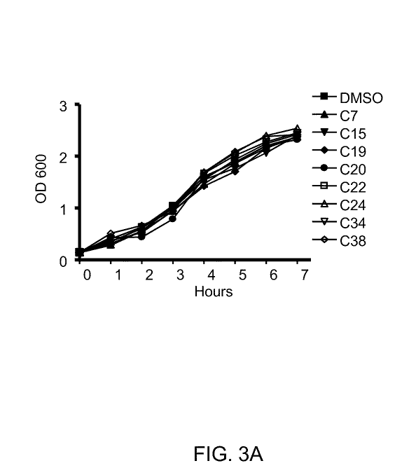

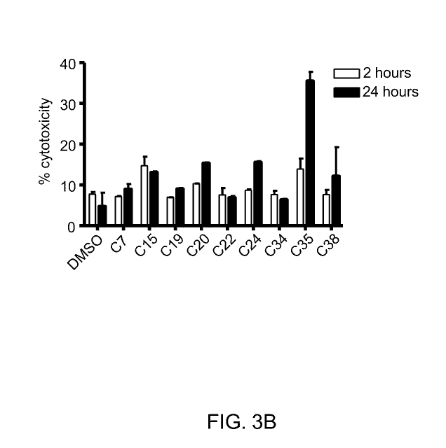

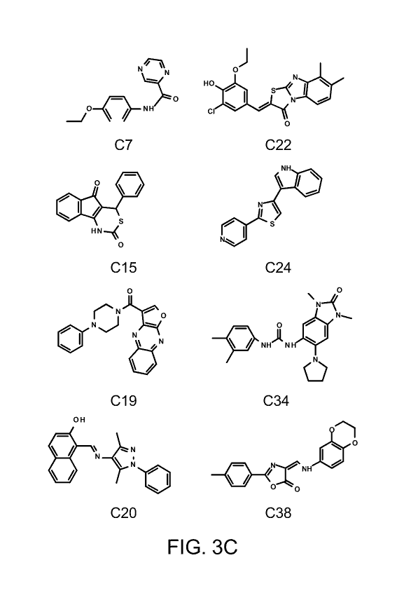

The remaining 12 compounds were evaluated for toxicity to either Yptb or HEp-2 cells. To determine if the compounds inhibited bacterial growth, Yptb was incubated at 26 C for 7 hrs in the presence of 60 μM of each compound. Nine of the compounds had no effect on bacterial growth under these conditions (FIG. 3A), while three proved to be antibacterial. The 9 non-antibacterial compounds were assessed for their ability to cause damage to epithelial cells by monitoring the release of lactate dehydrogenase (LDH), a cytoplasmic enzyme whose presence in cell supernatants indicates membrane damage. HEp-2 cells were incubated with 60 μM of each compound, in the absence of Yptb. Tissue culture supernatants were collected after 2 or 24 hours and assessed for LDH levels. Two compounds, C15 and C35, caused elevated LDH release at 2 hours (FIG. 3B). C15 did not cause further membrane damage between 2 and 24 hours exposure. In contrast, C35 had above baseline LDH release levels at 2 hours and this effect was exacerbated at 24 hours (FIG. 3B). Compounds C20 and C24 caused slightly elevated LDH release at 24 hours. Because of the high-level of toxicity caused by C35 at both time points, it was excluded from further characterization. In all subsequent experiments with the remaining 8 compounds, HEp-2 cells were exposed to compounds for 2 hours or less. The structures of the 8 compounds characterized are shown in FIG. 3C.

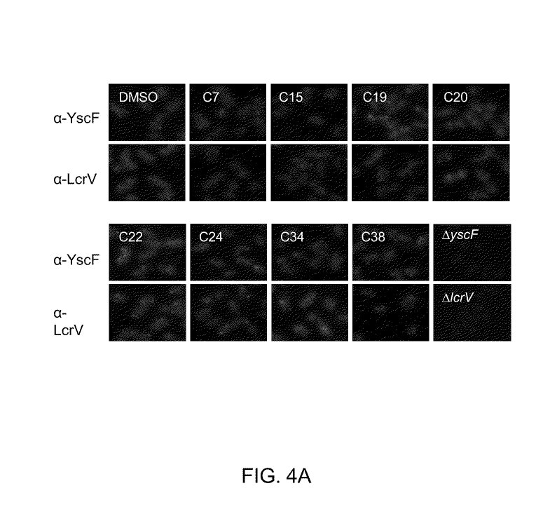

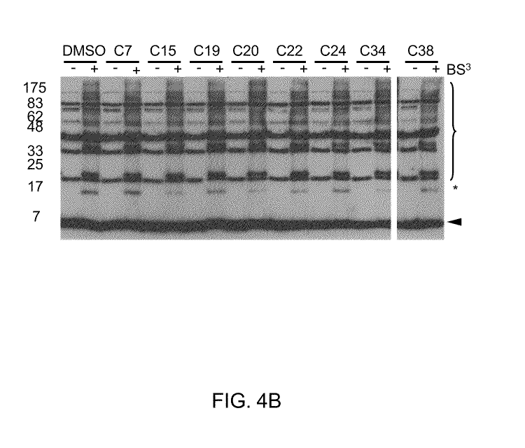

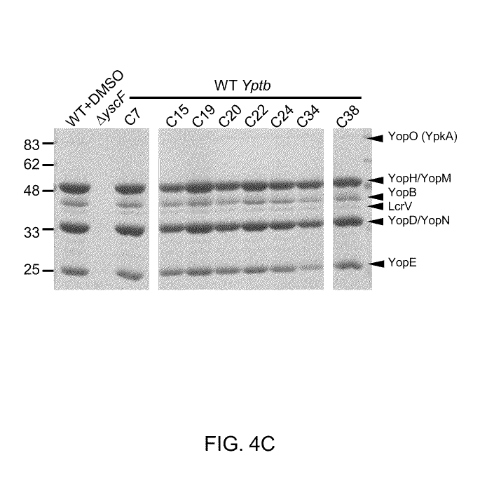

Inhibition of cell rounding in the presence of these molecules could be due to the inability of Yptb to form a functional TTSS needle, which is essential for the translocation of YopE. To assess whether the TTSS was assembled on the surface of Yptb, the compounds were tested to determine whether they disrupted needle architecture. First, the presence of YscF on the surface of Yptb was assayed by immunofluorescence with anti-YscF antibody. Yptb were grown in 5 mM CaCl₂ at 37 C with 60 μM of the indicated compound or DMSO for 1.5 hours. Yptb were mounted and fixed on coverslips and labeled with α-YscF or α-LcrV antibody, then visualized with Alexa fluor 594 conjugated α-rabbit antibody (red stain). Coverslips were counter stained with DAPI (blue stain). Images were pseudocolored and merged in MetaVue.Surface-localized YscF, appears as dots that surround the bacterium (FIG. 4A, DMSO α-YscF). Staining with anti-YscF antibody revealed that YscF was associated with Yptb after growth in all compounds tested (FIG. 4A). YscF staining was reduced in C24 and C38 treated bacteria.

To determine if the compounds affected the structure of the needle, chemical crosslinking analysis was performed on bacteria grown in the presence of compounds. Yptb, grown in 3 mM CaCl₂ and 60 μM compounds or 0.3% DMSO, were treated with 1 mM BS³ or water. Yptb were solublized and Western blot analysis was performed with α-YscF antibody. The asterisk shows YscF dimer, the arrowhead denotes YscF monomer and the bracket indicates high molecular weight YscF polymers. BS³, a membrane impermeable crosslinker, covalently links YscF lysine residues between neighboring YscF molecules in the assembled needle, resulting in a characteristic laddering pattern observed by western analysis with YscF antibody (FIG. 4B, DMSO+BS³) (Ferracci, F. et al., 2005. Mol. Microbiol., 57:970-87). None of the compounds led to major changes in the cross-linking pattern of the YscF polymer indicating that the general structure of the needle was not significantly affected by the compounds. There was, however, a slight difference in the structure of the needles formed in the presence of C34. The crosslink of two YscF monomers (as indicated by the asterisk in FIG. 4B) was apparent in Yptb exposed to DMSO only. In the presence of C34, the crosslink of two monomers was consistently weaker. C15 and C20 also appear to have a weaker dimer crosslink. Together the immunofluorescence and crosslinking data indicate that YscF was polymerized on the surface and formed needles that were not detectably different in the presence of compounds compared to needles formed in the absence of compounds.

LcrV has been observed at the tip of the needle and this localization is likely required for efficient pore-formation and subsequent translocation (Broms, J. et al., 2007. J. Bacteriol., 189:8417-29; Broz, P. et al., 2007. Mol. Microbiol., 65:1311-20). Therefore, destabilization of LcrV at the tip could lead to reduced translocation. The association of LcrV with the exterior of Yptb was tested by immunofluorescence with LcrV antibodies (FIG. 4A, DMSO α-LcrV). Similar to α-YscF staining, surface-localized LcrV appear as dots surrounding the bacterium. LcrV associated with Yptb in the presence of 60 μM of each compound (FIG. 4A), although the overall levels of fluorescence for Yptb grown in C24 and C38 were slightly reduced consistent with lower levels of YscF staining. These data suggest that although there may be some differences in the architecture of the needles in the presence of C34, the defects in translocation the compounds caused were not due to inability of Yptb to form needles or an inability of LcrV to localize on the surface.

To evaluate the ability of the compounds to block secretion, Yptb were grown at 37 C in the absence of calcium, conditions which permit high levels of Yop secretion into culture supernatants (Yother, J. & J. Goguen, 1985. J. Bacteriol., 164:704-11). A strain lacking YscF (FIG. 4C, ΔyscF) is incapable of secreting Yops, and served as a negative control for secretion. The ability to secrete Yops was not impeded by the compounds (FIG. 4C), with the exception of C34, which consistently secreted less, but detectable levels of Yops. These results, combined with the immunofluorescence and crosslinking data, suggest that these compounds do not disrupt the ability of Yptb to form a Yop secretion-competent TTSS.

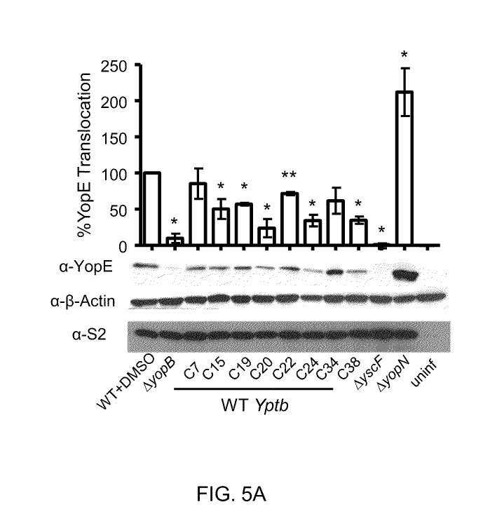

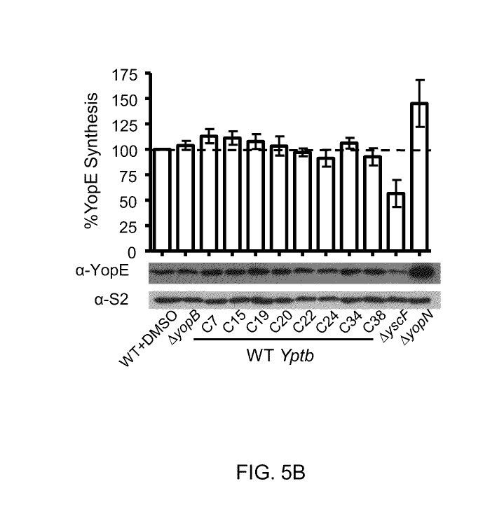

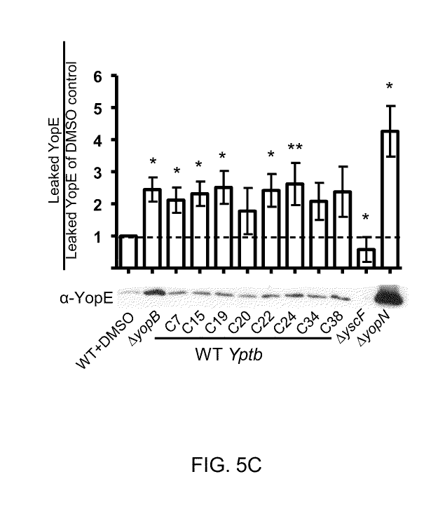

The ability of the compounds to reduce cell-rounding was evaluated to determine whether it was due to a defect in translocation of YopE, the effector responsible for this phenotype. HEp-2 cells were infected in the presence of compounds or DMSO and after 45 minutes the amount of YopE translocated into HEp-2 cells was determined by western analysis. Translocated YopE protein levels were normalized to both the total amount of bacteria (FIG. 4A, α-S2) and the total amount of HEp-2 cells loaded (FIG. 4A, α-β-Actin). The yopB and yscF mutants lack necessary components for translocation and secretion, respectively, and as expected were defective for translocation (FIG. 5A, ΔyopB, ΔyscF). A strain carrying a deletion of the regulatory protein, YopN, which hypersecretes Yops into culture supernatants, also hypertranslocated Yops into target cells compared to cells infected with WT (FIG. 5A, ΔyopN). Analysis revealed that 6 of the 8 compounds significantly reduced YopE translocation into the cytosol of HEp-2 cells (FIG. 5A), consistent with the defects in cell-rounding (FIG. 2A). C7 did not inhibit translocation of YopE into HEp-2 cells, supporting the observation that the cell-rounding defect was weak, and therefore the difference in translocation of YopE may not be strong enough to detect in this assay. C34 also did not inhibit translocation of YopE, but was a potent inhibitor of cell-rounding. This suggests that C34 may target other factors that influence cell-rounding that do not interfere with translocation.

A decrease in translocation could result from several defects including expression of Yops, sensing cell contact to trigger Yop translocation, adherence of Yptb to target cells, leading to faulty pore-formation. To determine whether the compounds reduced the level of Yop synthesis, the total levels of YopE in Yptb was assayed during infection by Western analysis (FIG. 5B). The yscF mutant synthesized fewer Yops, due to either downregulation or a failure to upregulate synthesis after cell contact because it cannot secrete Yops, while the yopN mutant produced higher levels of YopE. The levels of YopE during infection in the presence of compounds were comparable to the DMSO control for all compounds tested, indicating that the failure to translocate Yops was not due to a defect in Yop synthesis.

Lowered levels of translocated YopE could be caused by an inability to efficiently transfer YopE in a polarized manner into HEp-2 cells. Some mutants in the TTSS with low levels of translocation leak excessive Yops into culture supernatants during infection. To assess whether these compounds led to inefficient transfer of YopE into host cells, the presence of YopE in the media of infected HEp-2 cells was determined (FIG. 5C). Infection with ΔyopB is Yptb led to an excess of YopE leaked into supernatants, and higher levels of YopE were detected in supernatants of the HEp-2 cells infected with ΔyopN. Infection with WT Yptb in the presence of C7, C15, C19, C22, and C24 caused significant leakage Yops into the culture supernatants, as compared to DMSO treated controls. The remaining compounds, C20, C34, and C38 also consistently caused elevated levels of Yop leakage. These results suggest that the compounds interfere with the transfer of Yops into host cells. Alternatively, the compounds could be causing aberrant secretion of Yops into tissue culture supernatants independent of cell contact. To test this possibility, Yptb was grown under conditions non-permissive for Yop secretion in the presence of the compounds and found that none of the compounds induced Yop secretion in the absence of HEp-2 cells. These results indicate that the compounds interfere with the efficient polarized translocation of YopE, resulting excessive leakage of Yops into culture supernatants during infection. In addition, these results suggest that the Yptb retain the ability to sense cells and to trigger the process of polarized translocation of Yops from Yptb into target cells.

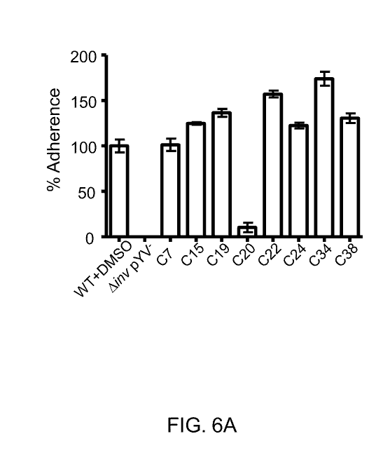

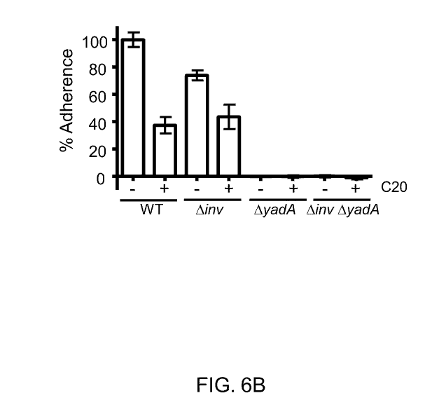

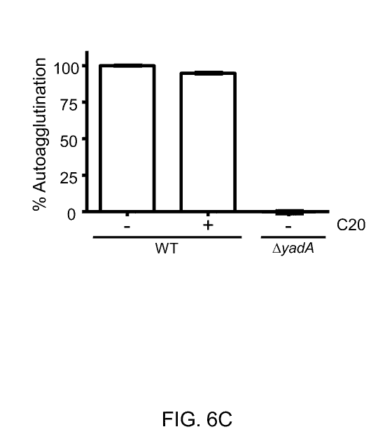

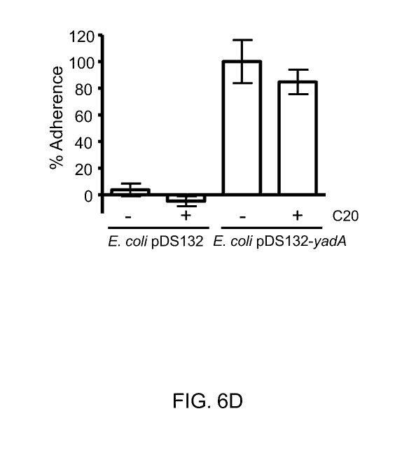

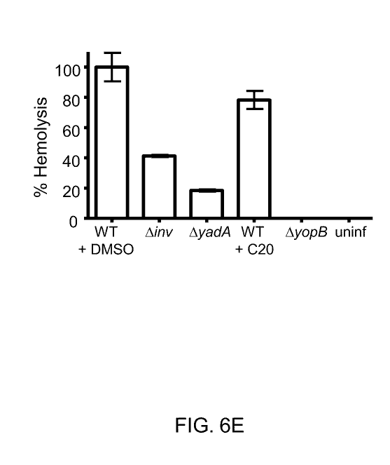

Adherence of Yptb to host cells is essential for the translocation of Yops. To test if compounds reduced adherence of Yptb to HEp-2 cells, monolayers of HEp-2 cells were infected with Yptb in the presence of compounds. Adherent Yptb can be detected with sera raised against whole Yersinia and the amount of bound Yptb determined by enzyme-linked immunosorbant assay (ELISA). One compound, C20, significantly reduced binding of Yptb to HEp-2 cells to 15-20% of the levels of the DMSO-treated control (FIG. 6A). Most compounds did not interfere with binding suggesting that the translocation defect caused by the remainder of the compounds was not due to inefficient adherence of Yptb to target cells.

Two proteins, YadA and Invasin contribute to adherence of Yptb and delivery of Yops into HEp-2 cells. If C20 blocked adherence through either YadA or Invasin, then deletion of its target would prevent further interference of adherence mediated by other factors. Deletion of Invasin led to a small decrease in adherence of Yptb to HEp-2 cells but did not block the ability of C20 to further inhibit binding (FIG. 6B) indicating that the effect of C20 is not mediated through Invasin. In the absence of YadA, binding of Yptb to HEp-2 cells was not detected above background levels (FIG. 6B, ΔyadA and ΔyadA Δinv).

Since the presence of YadA was critical for adherence of Yptb to HEp-2 cells, YadA-mediated autoagglutination was assayed to determine whether it was affected by C20 (Skurnik, M. et al., 1984. J. Bacteriol., 158:1033-6). Yersinia expressing YadA protein can clump in dense culture through the interaction of the YadA moieties on neighboring cells. This interaction can be measured as a change in the OD between a settled culture and one that has been dispersed. The autoagglutination activity of Yptb was unaffected by C20 (FIG. 6C) indicating that C20 did not inhibit YadA-mediated autoagglutination. To test if C20 interfered with the binding of YadA-expressing bacteria that do not express other Yersinia adhesins, YadA was expressed in E. coli. E. coli harboring either pDS132-yadA or pDS132 alone were cultured in the presence or absence of C20, and the amount of bound E. coli was assessed by ELISA. E. coli expressing YadA bound to HEp-2 cells, while E. coli expressing vector alone did not (FIG. 6D). Incubation with C20 did not block the ability of E. coli expressing YadA to adhere to HEp-2 cells, suggesting that YadA was not the target of C20.

Translocon assembly and insertion of pore-forming proteins is essential for adequate translocation of Yops. The failure to properly adhere to cells may result in defective translocon insertion into the host cell plasma membrane, leading to defective translocation. To test whether adherence was required for pore-formation, the pore forming ability of Yptb with adherence defects was evaluated. Sheep red blood cells (SRBCs) were infected with strains of WT Yptb or strains lacking Invasin or YadA and pore-formation was detected by the release of hemoglobin. The percent hemolysis of SRBC infected with the Δinv mutant was reduced to 60% of WT controls, and the ΔyadA mutant was reduced 80% (FIG. 6E), indicating that adherence to SRBCs is critical for insertion of the pore. SRBCs infected with Yptb in the presence of C20 exhibited slightly reduced leakage of hemoglobin. This is consistent with the finding that C20 reduced adherence of Yptb to mammalian target cells, though the reduction in hemolysis is not as dramatic as the adhesin mutants. These data suggest that the requirement for adherence to HEp-2 cells and the requirement for adherence and/or translocon insertion are different between HEp-2 cells and SRBCs and that C20 interferes with factors that vary between the two cell types.

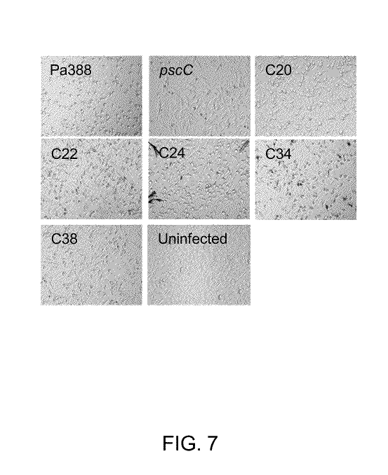

To determine whether the compounds reduced the translocation of effectors by other bacteria with closely related TTSSs, the compounds were evaluated to determine whether they inhibited translocation of ExoS from P. aeruginosa (Vallis, A. et al., 1999. Infect. Immun., 67:914-20). ExoS, like YopE, has a Rho-GAP activity that disrupts the actin cytoskeleton in cells targeted by Pseudomonas leading to rounding of cells (Krall, R. et al., 2002. Infect. Immun., 70:360-7). The P. aeruginosa strain, Pa388 (Yahr, T. et al., 1996. Mol. Microbiol., 22:991-1003), was used to infect HEp-2 cells in the presence of compounds and the degree of cell-rounding was observed by light microscopy (FIG. 7). Cultures of WT Pa388 or a translocation defective mutant (Pa388 pscC) were grown at 37 C in the presence of 60 μM compounds or 0.3% DMSO. The cultures were used to infect HEp-2 cells at an MOI of 10:1 in the presence of 60 μM compounds. The infection was allowed to proceed for 90 minutes at 37 C before imaging. HEp-2 cells infected with a pscC mutant, which lacks an essential component of the TTSS, remained flat—similar to uninfected cells (FIG. 7, pscC). Incubation with C7, C15 and C19 had little or no effect on ExoS-dependent cell rounding, while C20, C22, C24, C34, and C38, all reduced cell-rounding. These data demonstrate that several small molecules reduce translocation of TTSS effectors from other bacteria and suggest that these molecules target common features required for translocation.

### Discussion

HTS that target virulence factors have been used to identify novel small molecules that could be used as anti-infectives against pathogenic species of bacteria (Puri, A. & M. Bogyo, 2009. ACS Chem. Biol., 4:603-16). Small molecule anti-infectives are different from traditional antibiotics because they target factors important for the virulence of these organisms, but not viability. Thus, the target may avoid the rapid selective pressure that occurs with many other antibiotics. Several molecules that inhibit virulence factors have been identified in HTS, and some are effective in infection models (Felise, H. et al., 2008. Cell Host Microbe, 4:325-36; Hung, D. et al., 2005. Science, 310:670-4). For instance, Virstatin, is an inhibitor of V. cholerae ToxT, a transcriptional regulator of cholera toxin and toxin co-regulated pilus. Virstatin reduces the bacterial burden on mice infected with V. cholerae, without deleterious effects on bacterial growth. In another example, two compounds have been identified that inhibit intracellular trafficking of shigatoxin, ricin and diptheria toxin during specific stages of toxin translocation (Saenz, J. et al., 2007. Infect. Immun., 75:4552-61) leading to the diminished activity of these proteins. Additional HTS seeking to abrogate quorum sensing (QS) (Muh, U. et al., 2006. Antimicrob. Agents Chemother., 50:3674-9.) have led to the discovery of a specific homoserine lactone mimic, whose activity leads to down regulation of specific virulence factors in Pseudomonas (Muh, U. et al., 2006. Proc. Natl. Acad. ScL USA, 103:16948-52).

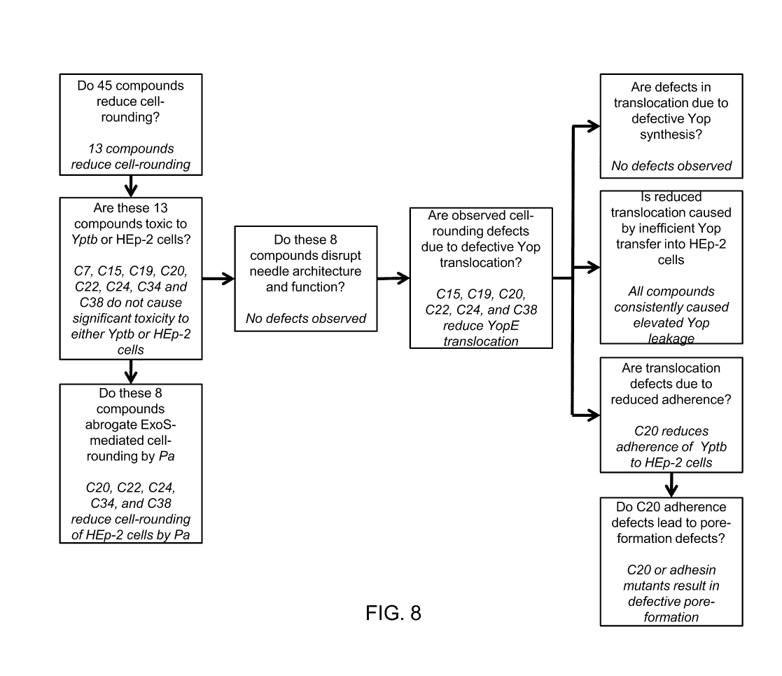

In addition to these general virulence factor inhibitor screens, several HTS have identified inhibitors of bacterial protein secretion systems important for virulence, including the type III (Aiello, D. et al., 2010. Antimicrob. Agents Chemother., 54:1988-99; Gauthier, A. et al., 2005. Antimicrob. Agents Chemother., 49:4101-9, Kauppi, A. et al., 2003. Chem. Biol., 10:241-9; Pan, N. et al., 2009. Antimicrob. Agents Chemother., 53:385-92) and type IV secretion systems (Charpentier, X. et al., 2009. PLoS Pathog., 5:e1000501). Inhibition of these protein secretion systems may block the ability of pathogenic organisms to deliver many virulence factors and thus be potent anti-infectives. The previous HTS screens involving TTSSs have identified molecules that inhibit the transcription or secretion of proteins from bacteria. Translocation of effector proteins into host cells is also important for virulence of organisms that rely on protein secretion systems, and thus may be an important target for the identification of novel classes of inhibitors. In contrast, described herein is a screen where 13 compounds were identified that inhibits translocation of the E-TEM fusion protein into mammalian cells (FIG. 8). Secondary assays revealed that six of the molecules specifically interfere with translocation of effectors without blocking protein synthesis or secretion of effectors in vitro or causing toxicity to Yptb or HEp-2 cells (FIG. 8). They were structurally distinct yet all were small, planar, and hydrophobic. Given this similarity, it is possible that these molecules disrupt hydrophobic interactions occurring at the membrane between Yptb and the target cell.

Events that are critical for Yop translocation but not secretion include the ability to sense cell contact, form pores, adhere to host cells, and activate host cell signal-transduction cascades (Fallman, M. & A. Gustaysson, 2005. Int. Rev. Cytol., 246:135-88). The result that Yops flux through the base and needle after host cell contact, but are leaked into the extracellular space rather than translocated into host cells indicates that many of these compounds act at the interface between the TTSS and the host cell (FIG. 8). Moreover, these results suggest that the compounds do not prevent sensing of host cells since secretion is triggered upon cell contact. Likewise, since LcrV remains situated on the tip of the TTSS when compounds are present, the compounds do not disrupt its association with needles (FIG. 8). Excessive leakage of Yops, however, may result from a failure of YopB, YopD and LcrV to form a functional pore through their presumed interaction, a failure of YopB and/or YopD to properly insert into membranes, or a disruption of one or more host factors required for adequate pore formation or translocation.

Another key requirement for translocation but not secretion is adherence of Yptb to host cells. The fact that C20 interferes with bacterial adherence likely contributes to the observed translocation defect (FIG. 8). C20 also had a small but reproducible defect in SRBC hemolysis, which is consistent with the result that C20 disrupts adherence leading to inefficient translocon insertion and effector translocation. C20, however, did not appear to interfere with YadA or Invasin function. C20 may interfere with another adhesin; it may alter other bacterial membrane properties, such as LPS, which in turn reduces adherence, or it may reduce/modify host cell receptors or host membrane characteristics that are necessary for tight interactions between bacteria and host cells. In fact, the observation that C20 also reduces translocation by P. aeruginosa suggests that C20 targets a similar factor or mechanism conserved between both organisms (FIG. 8).

The finding that C20, C22, C24, C34 and C38 all demonstrate diminished ExoS-mediated cell-rounding in HEp-2 cells suggests that these compounds target host cell factors or that they target conserved bacterial elements between the closely related TTSS of Yersinia and P. aeruginosa, including the homologous translocon components. There is precedent for regulation of translocation by host cell factors (Aili, M. et al., 2008. Int. J. Med. Microbiol., 298:183-92). Upon interaction of Yersinia adhesins with host cell integrins, Src kinase activation leads to enhanced translocation. Additionally, bacterial contact with lipid rafts has been implicated in triggering of type three secretion in Shigella flexneri (van der Goot, F. et al., 2004. J. Biol. Chem., 279:47792-8). It is possible that C22, C24, C34 and C38 alter one or more of these factors. Conversely, the observation that several other compounds, C7, C15 and C19 have no discernable effects on P. aeruginosa mediated cell-rounding suggests that these compounds target mechanisms specific to Yptb. C34 causes slight differences in structure of the needle, reduces levels of secretion and dramatically inhibits cell-rounding, all without reducing translocated YopE. These results suggest that C34 has multiple targets including a host factor that reduces the cell rounding activity caused by YopE or conversely, that C34 directly inhibits YopE activity.

One or more of these compounds may act on the host cell membrane and/or host cell factors required for translocation. LDH data indicated that one compound did indeed affect membrane permeability after extensive incubation with compounds, but most of these compounds had no discernible effect on LDH release. Similarly, none of the compounds appeared to disrupt actin filaments, suggesting that RhoA was still active and active RhoA is critical for Yop translocation. One or more of these compounds could, however, target host cell factors or membranes critical for translocation and have only subtle effects on cell shape or membrane permeability. A recent screen with Legionella, for example, identified 22 compounds that reduced translocation by type IV secretion into J774 cells. These compounds target various host cell processes including cytoskeleton proteins and proteins involved in cytoskeleton dynamics, as well as surface proteins that may be involved in binding and internalization of Legionella but they did not all have gross effects on cell morphology.

There have been studies designed to identify inhibitors of type three mediated effector secretion in various types of bacteria, including Yptb, Y. pestis, Chlamydia, Salmonella typhimurium, P. aeruginosa and enteropathogenic E. coli (EPEC) (Bailey, L. et al., 2007. FEBS Lett., 581:587-95; Hudson, D. et al., 2007. Antimicrob. Agents Chemother., 51:2631-5; Muschiol, S. et al., 2006. Proc. Natl. Acad. Sci. USA, 103:14566-71; Negrea, A. et al., 2007. Antimicrob. Agents Chemother., 51:2867-76; Nordfelth, R. et al., 2005. Infect. Immun., 73:3104-14; Veenendaal, A. et al., 2009. J. Bacteriol., 191:563-70; Williamson, E. et al., 1995. FEMS Immunol. Med. Microbiol., 12:223-30). The best-studied inhibitors were first identified by Kauppi et al., in a screen that monitored YopE promoter activity. This family of molecules appears to inhibit type three secretion in a variety of pathogens, including Chlamydia, Shigella and Salmonella. Another study identified molecules that permitted Yptb growth under Yop-inducing conditions, which are normally restrictive to growth. Several of these molecules inhibited both Yop secretion and secretion of type three secretion-related proteins from EPEC. Two additional studies screened for molecules that reduced effector secretion from EPEC or Salmonella. In the EPEC study, one compound inhibited expression of type three secretion-related proteins but not the expression of other non-type three secretion proteins. The Salmonella study also identified many compounds that inhibited protein expression. One of the compounds identified however, did not inhibit protein expression or growth of bacteria, but reduced secretion of effectors from several TTSSs and interfered with assembly of the needle complex. While these molecules are all potentially powerful molecules, and some reduce virulence in infection model systems, the protein targets of these molecules have not been identified.

### Other Embodiments

Other embodiments will be evident to those of skill in the art. It should be understood that the foregoing detailed description is provided for clarity only and is merely exemplary. The spirit and scope of the present disclosure are not limited to the above examples, but are encompassed by the following claims. The contents of all references cited herein are incorporated by reference in their entireties.

What is claimed is:

1. A method of treating or preventing an infection comprising administering to a subject at risk for, diagnosed with, or exhibiting symptoms of an infection a compound that inhibits Yop translocation.

2. The method of claim 1, wherein the compound is selected from the group consisting of: N-(4-ethoxyphenyl)pyrazine-2-carboxamide, (C7); 4-phenyl-1,4-dihydroindeno[1,2-d][1,3]thiazine-2,5-dione, (C15); furo[3,2-b]quinoxalin-3-yl-(4-phenylpiperazin-1-yl)methanone, (C19); 1-[(E)-(3,5-dimethyl-1-phenyl-pyrazol-4-yl)iminomethyl]naphthalen-2-ol, (C20); (2Z)-2-[(3-chloro-5-ethoxy-4-hydroxy-phenyl)methylene]-5,6-dimethyl-thiazolo[3,2-a]benzimidazol-1-one, (C22); 4-(1H-indol-3-yl)-2-(4-pyridyl)thiazole, (C24); 1-(1,3-dimethyl-2-oxo-6-pyrrolidin-1-yl-benzimidazol-5-yl)-3-(3,4-dimethylphenyl)urea, (C34); and (4E)-4-[(2,3-dihydro-1,4-benzodioxin-6-ylamino)methylene]-2-(p-tolyl)oxazol-5-one (C38) and salts, derivatives and substituted structures thereof.

3. The method of claim 1, wherein the infection is from a pathogen comprising a TTSS.

4. The method of claim 3, wherein the pathogen is a gram negative bacterium.

5. The method of claim 3, wherein the pathogen is a Yersinia bacterium.

6. The method of claim 1, wherein the compound inhibits Yop translocation without affecting synthesis of TTSS components.

7. The method of claim 1, wherein the subject is a mammal.

8. The method of claim 6, wherein the mammal is a human.

9. A pharmaceutical composition comprising a compound is selected from the group consisting of: N-(4-ethoxyphenyl)pyrazine-2-carboxamide, (C7); 4-phenyl-1,4-dihydroindeno[1,2-d][1,3]thiazine-2,5-dione, (C15); furo[3,2-b]quinoxalin-3-yl-(4-phenylpiperazin-1-yl)methanone, (C19); 1-[(E)-(3,5-dimethyl-1-phenyl-pyrazol-4-yl)iminomethyl]naphthalen-2-ol, (C20); (2Z)-2-[(3-chloro-5-ethoxy-4-hydroxy-phenyl)methylene]-5,6-dimethyl-thiazolo[3,2-a]benzimidazol-1-one, (C22); 4-(1H-indol-3-yl)-2-(4-pyridyl)thiazole, (C24); 1-(1,3-dimethyl-2-oxo-6-pyrrolidin-1-yl-benzimidazol-5-yl)-3-(3,4-dimethylphenyl)urea, (C34); and (4E)-4-[(2,3-dihydro-1,4-benzodioxin-6-ylamino)methylene]-2-(p-tolyl)oxazol-5-one (C38) and salts, derivatives and substituted structures thereof.

10. The pharmaceutical composition of claim 8, further comprising a second anti-bacterial agent.

11. A method of identifying anti-bacterial activity of a compound comprising:
a) mixing a sample comprising a test compound, a Yop, and a cell; and
b) measuring inhibition of Yop translocation into the cell,
wherein a statistically relevant inhibition of Yop translocation is indicative of the anti-bacterial activity of the compound.

12. A method of identifying a compound that inhibits Yop translocation into a cell comprising:
a) incubating a recombinant bacterial strain that expresses a chimeric protein comprising a Yop sequence and exogenous sequence, and a cell comprising a detectably labeled reporter, wherein the reporter alters its signal in the presence of the exogenous sequence of the chimeric protein, in the presence or absence of a test compound under conditions that allow for translocation of the chimeric protein into the cell in the absence of a test compound; and
b) detecting the reporter to determine if it is in the presence of the chimeric protein;
wherein if the reporter is not in the presence of the chimeric protein, then the test compound inhibits Yop translocation.

13. The method of claim 12, wherein the reporter is fluorescently labeled.

14. The method of claim 13, wherein the reporter comprises two fluorescent labels that form a fluorescent resonance energy transfer pair, wherein the first fluorescent label emits energy at the excitation wavelength of the second fluorescent label.

15. The method of claim 14, wherein the two fluorescent labels are connected by a lactam ring.

16. The method of claim 15, wherein the two fluorescent labels are coumarin and CCF2.

17. The method of claim 12, wherein the Yop sequence is the N-terminal 100 amino acids of YopE.

18. The method of claim 12, wherein the exogenous sequence of the chimeric protein comprises an enzymatic activity that modifies the reporter.

19. The method of claim 18, wherein the enzymatic activity is a lactamase activity.

20. A kit comprising a pharmaceutical composition comprising a compound is selected from the group consisting of: C7, C15, C19, C20, C22, C24, C34 and C38, and salts, derivatives and substituted structures thereof, and one or more reagents for administering the pharmaceutical composition to a subject.

21. The method of claim 1, wherein the compound is administered in combination with a second antibacterial agent.
